(19) United States
(12) Patent Application Publication
(10) Pub. No.: US 2008/0027006 A1
(43) Pub. Date: Jan. 31, 2008

(54) Compositions And Methods For Modification And Prevention Of Sars Coronavirus Infectivity

(75) Inventors: Brian Tripet, Bozeman, MT (US); Robert S. Hodges, Denver, CO (US)

Correspondence Address: GREENLEE WINNER AND SULLIVAN P C, 4875 PEARL EAST CIRCLE, SUITE 200, BOULDER, CO, 80301, US

(73) Assignee: The Regents of the University of Colorado, Boulder, CO (US)

(21) Appl. No.: 10/597,914

(22) PCT Filed: Feb. 14, 2005
(86) PCT No.: PCT/US05/04408

§ 371 Date: Aug. 3, 2007

Related U.S. Application Data
(60) Provisional application No. 60/544,410, filed on Feb. 12, 2004.

(51) Int. Cl. A61K 38/10; A61P 31/00; C07K 7/08; C12Q 1/70

(52) U.S. Cl. 514/14; 435/5; 530/327

(57) ABSTRACT

The invention relates to diseases of coronaviruses, particularly severe acute respiratory syndrome (SARS). Compositions and methods in connection with peptide-based compounds are described relating to inhibition or prevention of coronavirus activity including that of SARS coronavirus and the ability of its S protein to achieve fusion with and entry into a target cell.

### CROSS-REFERENCES TO RELATED APPLICATIONS

This application claims the benefit of U.S. Provisional Application No. 60/544,410, filed Feb. 12, 2004, which is incorporated herein by reference in entirety.

### STATEMENT ON FEDERALLY SPONSORED RESEARCH OR DEVELOPMENT

This invention was made with government support under Grant/Contract Nos. NIH R01AI48717, NIH R01AI31948, and NIH R01AI25231 awarded by the National Institutes of Health. The government has certain rights in the invention.

### BACKGROUND OF THE INVENTION

In the fall of 2002, the World Health Organization witnessed an outbreak of atypical pneumonia termed severe acute respiratory syndrome (SARS) which quickly spread to over 25 countries world-wide. The causative agent of SARS was quickly identified to be a previously unknown member of the family of coronaviruses (1-4). The coronaviruses are a diverse group of large enveloped, positive-stranded RNA (ss RNA) viruses that cause respiratory and enteric diseases in humans and other animals. For example, previously known human coronaviruses, HCoV-229E and HCoV-OC43 cause up to 30% of common colds, and rarely cause pneumonia in older adults or immunocompromised patients (5-7). Coronaviruses of animals (e.g., porcine transmissible gastroenteritis virus (TGEV), murine hepatitis virus (MHV) and avian infectious bronchitis virus (IBV)) cause respiratory, gastrointestinal, neurological, or hepatic disease in their respective hosts (8). In the case of the SARS-associated coronavirus (SARS-CoV), the genomic nucleotide sequence shows closest identity to novel coronavirus strains isolated from Himalayan palm civets and a raccoon dog in the Guangdong province of China (9, 10). The human isolate of SARS-COV can also grow in monkeys, mice, cats and ferrets.

Coronavirus infection is achieved through fusion of the lipid bilayer of the viral envelope with host cell membranes. Membrane fusion is mediated by the viral spike (S) glycoprotein on the viral envelope (11-14). The S-glycoprotein is synthesized as a precursor of about 180 kDa that oligomerizes in the endoplasmic reticulum and is incorporated into budding virions in a pre-Golgi compartment. In some strains, S is cleaved by trypsin or related enzymes to yield two non-covalently associated subunits: S1 and S2 (15, 16). S1 contains the receptor-binding site and thus contributes to defining the host range of the virus (17, 18). S2 is the transmembrane subunit which contributes to mediating fusion between viral and cellular membranes. S2 contains two 4,3-hydrophobic repeat domains (HR) that are predicted to form coiled-coil structures (14, 19). These regions are denoted HR-N and HR-C, and are separated by an intervening stretch of about 140 amino acid residues called the interhelical domain. These coiled-coil regions may play an important role in defining the oligomeric structure of the spike protein in its native state and its fusogenic ability (20). The presence of the HR regions in conjunction with recent studies by Bosch et al., (19) indicate that coronavirus spike proteins can be classified as type 1 viral fusion proteins.

There is a pressing need for effective anti-viral approaches in connection with SARS, such as therapeutic treatment, prevention, and diagnosis. The ability of viruses such as the SARS virus and other coronaviruses to mediate fusion and thus achieve infection of a host cell and spread presents an attractive target for defensive approaches.

### SUMMARY OF THE INVENTION

Abbreviations/symbols: SARS; severe acute respiratory syndrome; HR, hydrophobic repeat; HR-N, amino terminal hydrophobic repeat domain; HR-C; carboxy terminal hydrophobic repeat domain; MHV, mouse hepatitis virus; TFA, trifluoroacetic acid; SDS-PAGE, sodium dodecylsulfate-polyacrylamide electrophoresis; TFE, trifluroethanol; RP-HPLC, reversed-phase high performance liquid chromatography; θ, theta; Aib, aminoisobutyric acid; Dxg, dipropyl or dibutyl glycine; DIC, diisopropylcarbodiimide, Gdn-HCl, guanidinium hydrochloride; KLH, keyhole limpet hemocyanin; BSA, bovine serum albumin; SEC, size exclusion chromatography.

In general the terms and phrases used herein have their art-recognized meaning, which can be found by reference to standard texts, journal references and contexts known to those skilled in the art. The following definitions are provided to clarify their specific use in the context of the invention.

When used herein, the term “target cell” refers to a cell that can be susceptible to entry or infection by a virus. For example, a target cell can be susceptible to a coronavirus or in particular a SARS coronavirus. In an embodiment a target cell is mammalian. In a preferred embodiment a target cell is human.

When used herein, the term “peptide” refers to a peptide, polypeptide, or protein. The term can encompass a natural peptide such as a fragment of a native S protein of a SARS-coronavirus. The term also encompasses a variant peptide which can be other than a natural peptide, such as a substituted or derivatized fragment of a native S protein. Thus the term is intended to encompass any natural and variant peptides and derivatives thereof, and the inclusion of a descriptor such as natural or variant is used for convenience or clarity for context. The term can relate to a peptide sequence or a peptide product. For example, the term can refer to an HR-N10 peptide with a lactam bridge or a mutated/substituted HR-N10 analog.

When used herein, the term “vector” refers to a molecular biology tool for use in a recombinant expression system. In a particular embodiment, a vector comprises a deoxynucleotide gene sequence and is adapted for expression in a given host cell of an organism such as a bacterium, yeast, insect, or mammal.

When used herein, the term “insert” refers to a sequence that can be used in connection with a vector. A vector with an insert can be used in a recombinant expression system.

When used herein, the term “effective amount” refers to an amount capable of achieving at least a partial result. For example, an effective amount of a peptide can effect inhibition of viral entry either partially, substantially, or completely. In a particular example, an effective amount of a peptide inhibitor effects reduced infectivity of a SARS coronavirus.

When used herein, the term “benign” refers to a medium, buffer, or conditions that are typical for or compatible with a eukaryotic cell.

When used herein, the term “derivatized amino acid” refers to any amino acid that is derivatized chemically or biosynthetically.

When used herein, the term “unnatural amino acid” refers to a synthetic amino acid or refers to an amino acid that is typically foreign to a particular organism. Unnatural amino acids can optionally be a subset of non-proteinogenic amino acids.

In an embodiment, the invention provides a composition comprising a purified peptide of a SARS coronavirus S protein, wherein said peptide is capable of modification of SARS coronavirus infectivity. In an embodiment, said modification is an inhibition of infectivity. In an embodiment, the peptide has a conformational constraint, wherein said constraint enhances an ability to maintain an alpha-helical conformation. In an embodiment, the peptide comprises a lactam bridge, salt bridge, or other covalent bond.

In an embodiment, the invention provides a purified peptide selected from the group consisting of: SEQ ID NOS: 4, 6, 8, 10, 12, 14, 16, 18, 20, 22, 24, 26, 28, 30, 32, 34, 36, 38, 40, 42, 44, 46, 47, 48, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, and 102.

In an embodiment, the invention provides an HR-N peptide selected from the group consisting of HR-N10 (SEQ ID NO:24) and HR-N10a (SEQ ID NO:47). In an embodiment, the invention provides an HR-C peptide selected from the group consisting of HR-C4 (SEQ ID NO:46) and HR-C4a (SEQ ID NO:48).

In an embodiment, the invention provides a composition comprising a purified peptide of a SARS coronavirus S protein, wherein said peptide is capable of modifying an ability of said S protein to form or maintain a conformation relating to fusion or entry in a target cell.

In an embodiment, the invention provides a composition comprising a purified peptide HR-N10 (SEQ ID NO:24) or HR-N10a (SEQ ID NO:47). In an embodiment, the invention provides a composition comprising a purified peptide HR-C4 (SEQ ID NO:46) or HR-C4a (SEQ ID NO:48).

In an embodiment, the invention provides a composition comprising an alpha-helical trimeric conformation of an HR peptide of a coronavirus spike or fusion protein. In an embodiment, the invention provides a composition comprising an alpha-helical trimeric conformation of a purified HR peptide of a SARS coronavirus S protein. In a particular embodiment, said HR peptide is selected from the group consisting of HR-N10 (SEQ ID NO:24), HR-N10a (SEQ ID NO:47), HR-C4 (SEQ ID NO:46) and HR-C4a (SEQ ID NO:48). In a particular embodiment, said HR peptide is HR-N10 (SEQ ID NO:24) or HR-N10a (SEQ ID NO:47). In a particular embodiment, said HR peptide is HR-C4 (SEQ ID NO:46) or HR-C4a (SEQ ID NO:48).

In an embodiment, the invention provides a composition comprising an alpha-helical hetero-trimeric (6-helix) conformation of a purified HR peptide of a coronavirus spike or fusion protein. In an embodiment, the invention provides a composition comprising an alpha-helical hetero-trimeric (6-helix) conformation of a complex comprising a purified HR-N peptide and a purified HR-C peptide of a SARS coronavirus S protein. In a particular embodiment, the HR-N peptide is selected from the group consisting of HR-N1 (SEQ ID NO:6), HR-N2 (SEQ ID NO:8 ), HR-N10 (SEQ ID NO:24), and HR-N10a (SEQ ID NO:47); and said HR-C peptide is selected from the group consisting of HR-C1 (SEQ ID NO:40), HR-C4 (SEQ ID NO:46), and HR-C4a (SEQ ID NO:48). In a particular embodiment, the HR-N peptide is HR-N10 (SEQ ID NO:24) or HR-N10a (SEQ ID NO:47) and said HR-C peptide is HR-C4 (SEQ ID NO:46) or HR-C4a (SEQ ID NO:48). In an embodiment, the HR-N peptide is HR-N10 (SEQ ID NO:24) or HR-N10a (SEQ ID NO:47) and said HR-C peptide is HR-C1 (SEQ ID NO:40).

In an embodiment, the invention provides a composition comprising a purified HR-N or HR-C peptide of SARS coronavirus S protein, wherein said peptide is capable of modification of SARS coronavirus infectivity and wherein said peptide has a transition midpoint temperature of from about 35 to about 90 degrees Celsius. In an embodiment, said temperature is from about 36 to about 74 degrees Celsius. In an embodiment, said temperature is from about 37 to about 60 degrees Celsius. In an embodiment, said temperature is from about 56 to about 57 degrees Celsius.

In an embodiment, the invention provides a purified peptide comprising at least about 14 contiguous amino acids derived from a peptide selected from the group consisting of: HR-N10 (SEQ ID NO:24), HR-N10a (SEQ ID NO:47), HR-C4 (SEQ ID NO:46), and HR-C4a (SEQ ID NO:48), wherein said peptide exhibits a stable helix conformation at a physiological temperature of a human or other mammal.

In an embodiment, the invention provides a composition comprising a purified nucleic acid molecule encoding a peptide of SARS coronavirus S protein, wherein said peptide is capable of modification of SARS coronavirus infectivity. In an embodiment, the invention provides a composition comprising a purified nucleic acid molecule encoding an HR peptide of a SARS coronavirus S protein.

In an embodiment, the invention provides a composition comprising a purified nucleic acid molecule capable of encoding a peptide selected from the group consisting of: HR-N10 (SEQ ID NO:24), HR-N10a (SEQ ID NO:47), HR-C4 (SEQ ID NO:46), and HR-C4a (SEQ ID NO:48). In an embodiment, the invention provides a composition comprising a purified nucleic acid molecule capable of encoding peptide HR-N10 (SEQ ID NO:24) or HR-N10a (SEQ ID NO:47). In an embodiment, the invention provides a composition comprising a purified nucleic acid molecule capable of encoding peptide HR-C4 (SEQ ID NO:46) or HR-C4a (SEQ ID NO:48).

In an embodiment, the invention provides a method of identifying a peptide composition capable of inhibiting a SARS coronavirus, comprising: providing a peptide of an HR-N or HR-C domain of a SARS coronavirus S protein; and measuring an ability of said peptide to inhibit infectivity of a SARS coronavirus or cell fusion in connection with a SARS coronavirus protein; thereby identifying a peptide composition capable of inhibiting a SARS coronavirus.

In an embodiment, the invention provides a method of treating a SARS coronavirus infection in a human subject, comprising administering an effective amount of a peptide of a SARS coronavirus S protein.

In an embodiment, the invention provides a method of treating a SARS coronavirus infection in a human subject, comprising administering an effective amount of an antibody reactive towards a material selected from the group consisting of a SARS coronavirus S protein, a peptide of a SARS coronavirus S protein, an HR-N peptide of a SARS coronavirus S protein, an HR-C peptide of a SARS coronavirus S protein, HR-N10, HR-N10a, HR-C4, HR-C4a, HR-C1, a trimeric conformation of a SARS coronavirus peptide, a six helix bundle conformation of a complex of a SARS coronavirus HR-N peptide and an HR-C peptide. In an embodiment, said material is HR-N10 or HR-N10a. In an embodiment, said material is HR-C4 or HR-C4a. In an embodiment, said material comprises an HR-N peptide and an HR-C peptide. In an embodiment, said material comprises an HR-N peptide of HR-N10 or HR-N10a and an HR-C peptide of HR-C4 or HR-C4 a. In an embodiment, said material comprises an HR-N peptide of HR-N10 or HR-N10a and an HR-C peptide of HR-C1.

In an embodiment, the invention provides a SARS coronavirus purified peptide composition capable of stimulating an immune response, wherein said composition is selected from the group consisting of a SARS coronavirus S protein, a peptide of a SARS coronavirus S protein, an HR-N peptide of a SARS coronavirus S protein, an HR-C peptide of a SARS coronavirus S protein, HR-N10, HR-N10a, HR-C4, HR-C4a, HR-C1, a trimeric conformation of a SARS coronavirus peptide, a six helix bundle conformation of a complex of a SARS coronavirus HR-N peptide and an HR-C peptide.

In an embodiment, the invention provides a method of identifying or screening for an inhibitory peptide of a SARS coronavirus, comprising: providing a synthetic or recombinant peptide of HR-N or HR-C; evaluating said peptide for an ability to make a structure selected from the group consisting of an alpha-helical coil, an alpha-helical trimer, and an alpha-helical hetero-trimeric (6-helix) bundle; testing said peptide in an infectivity or cell-cell fusion bioassay, wherein a first result where said peptide is present and demonstrates a reduced infectivity or fusion in comparison with a second result where said peptide is absent; thereby identifying or screening for an inhibitory peptide.

The invention provides compositions comprising a peptide, a plurality of peptides, a complex of a peptide, or a complex of a plurality of peptides of a SARS coronavirus S protein, wherein said peptide, plurality of peptides, peptide complex, or complex of a plurality of peptides is capable of modification of SARS coronavirus infectivity and/or spread. In an embodiment, said modification is an inhibition of infectivity.

In an embodiment, said peptide has a conformational constraint, wherein said constraint enhances an ability to form or maintain an alpha-helical conformation. In an embodiment, said constraint enhances an ability to form or maintain a trimeric conformation, for example a homotrimer. In an embodiment, said constraint enhances an ability to form or maintain a six stranded bundle conformation, such as an alpha-helical hetero-trimeric (6-helix) conformation. In an embodiment, said peptide comprises a lactam bridge, a salt bridge, or other covalent bond.

In an embodiment, a composition comprises a nucleic acid molecule encoding a peptide of SARS coronavirus S protein, wherein said peptide is capable of modification of SARS coronavirus infectivity.

The invention provides a composition comprising peptide HR-N10. In an embodiment, a composition comprises a complex of peptide HR-N10. The invention provides a composition comprising peptide HR-C4. In an embodiment, a composition comprises a complex of peptide HR-C4. The invention provides a composition comprising peptide HR-N10 and peptide HR-C4. In an embodiment, a composition comprises a complex of peptide HR-N10 and peptide HR-C4.

In an embodiment, a composition comprises a single synthetic peptide molecule of an HR-N peptide and an HR-C peptide. In a particular embodiment, said single synthetic molecule comprises an HR-N10 peptide and an HR-C4 peptide, optionally separated by a spacer region.

In an embodiment, a composition comprises a nucleic acid molecule encoding a peptide of the invention. In a particular embodiment, said nucleic acid molecule encodes peptide HR-N10 or HR-C4. In an embodiment, a composition comprises a nucleic acid molecule encoding a plurality of peptides of the invention. In an embodiment, said nucleic acid molecule encodes an HR-N peptide and an HR-C peptide. In a particular embodiment, a composition comprises a nucleic acid molecule encoding peptide HR-N10 and HR-C4.

The invention provides a composition comprising an alpha-helical trimeric conformation of an HR peptide of a coronavirus spike or fusion protein. The invention provides a composition comprising an alpha-helical trimeric conformation of an HR peptide of a SARS coronavirus S protein. In an embodiment, said HR peptide is selected from the group consisting of HR-N10 and HR-C4. In an embodiment, said HR peptide is HR-N10 or peptide is HR-C4.

The invention provides a composition comprising an alpha-helical hetero-trimeric (6-helix) conformation of an HR peptide of a coronavirus spike or fusion protein. The invention provides a composition comprising an alpha-helical hetero-trimeric (6-helix) conformation of a complex comprising an HR-N peptide and an HR-C peptide of a SARS coronavirus S protein. In an embodiment, said HR-N peptide is selected from the group consisting of HR-N1, HRN-2, and HR-N10; and said HR-C peptide is selected from the group consisting of HR-C1 and HR-C4. In an embodiment, said HR-N peptide is HR-N10 and said HR-C peptide is HR-C4. In an embodiment, said HR-N peptide is HR-N10 and said HR-C peptide is HR-C1.

In an embodiment, the invention provides a composition consisting essentially of a peptide of the invention. In an embodiment, the invention provides a composition consisting essentially of a plurality of peptides of the invention. In an embodiment, the invention provides a composition consisting of a peptide of the invention. In an embodiment, the invention provides a composition consisting of a first and a second peptide of the invention.

The invention provides a composition comprising a nucleic acid molecule encoding an HR peptide of a SARS coronavirus S protein.

The invention provides a method of identifying a peptide composition capable of inhibiting a SARS coronavirus, comprising: providing a peptide of an HR-N or HR-C domain of a SARS coronavirus S protein; measuring an ability of said peptide to inhibit infectivity of a SARS coronavirus or cell fusion in connection with a SARS coronavirus protein; thereby identifying a peptide composition capable of inhibiting a SARS coronavirus.

The invention provides a method of treating a SARS coronavirus infection in a human subject, comprising administering an effective amount of a peptide of a SARS coronavirus S protein.

The invention provides an antibody to a material, where the material is: an HR-N peptide of a SARS coronavirus S protein; an HR-C peptide of a SARS coronavirus S protein; HR-N10; HR-C4; an alpha-helical trimeric conformation of an HR-N peptide, an HR-C peptide, HR-N10, or HR-C4; a six helix bundle conformation of an HR-N peptide and an HR-C peptide, HR-N10 and HR-C4, or HR-N10 and HR-C1; and a mixture of an HR-N peptide and an HR-C peptide, HR-N10 and HR-C4, or HR-N10 and HR-C1.

The invention provides a method of treating a SARS coronavirus infection in a human subject, comprising administering an effective amount of an antibody reactive towards a material selected from the group consisting of a SARS coronavirus S protein, a peptide of a SARS coronavirus S protein, an HR-N peptide of a SARS coronavirus S protein, an HR-C peptide of a SARS coronavirus S protein, HR-N10, HR-C4, HR-C1, a trimeric conformation of a SARS coronavirus peptide, a six helix bundle conformation of a complex of a SARS coronavirus HR-N peptide and an HR-C peptide. In an embodiment, said material is HR-N10. In an embodiment, said material is HR-C4. In an embodiment, said material comprises HR-N10 and HR-C4. In an embodiment, said material comprises HR-N10 and HR-C1.

The invention provides a SARS coronavirus peptide composition capable of stimulating an immune response, wherein said composition is selected from the group consisting of a SARS coronavirus S protein, a peptide of a SARS coronavirus S protein, an HR-N peptide of a SARS coronavirus S protein, an HR-C peptide of a SARS coronavirus S protein, HR-N10, HR-C4, and HR-C1; a trimeric conformation of a SARS coronavirus S peptide; a six helix bundle conformation of a complex of a SARS coronavirus HR-N peptide and an HR-C peptide; and a mixture of a SARS coronavirus HR-N peptide and an HR-C peptide. In an embodiment, a composition is an immunogenic composition or vaccine. In an embodiment, an immunogenic composition or vaccine is formulated or administered in connection with an adjuvant.

The invention provides a composition comprising a peptide of a SARS coronavirus S protein, wherein said peptide is capable of modifying an ability of said S protein to form or maintain a conformation relating to fusion or entry in a target cell.

In an embodiment, an individual peptide is used for inhibition of viral entry. In another embodiment, a plurality of peptides is used for inhibition of SARS viral entry. For example, a mixture of natural peptides HR-N10 and HR-C4 are used. Without wishing to be bound by a particular theory, the peptides are conceived to act by competitively binding with the corresponding sequences displayed in the native S2 domain of the SARS-CoV S protein, thereby blocking its ability to adopt the fusion active/competent state need for membrane fusion.

In an embodiment, a peptide is derived by one or more genetic engineering techniques such as recombinant expression, site-directed mutagenesis, or random mutagenesis. Other techniques are contemplated such as the use of an expanded genetic code; for example, advances in adapting the machinery for ribosomal protein biosynthesis in an expression system can allow integration of what would otherwise be non-proteinogenic amino acids.

In embodiments of the invention, a peptide comprises at least one chemical moiety such as a non-proteinogenic amino acid, unnatural amino acid, peptidomimetic unit, peptoid, beta amino acid, or derivatized amino acid. In a particular embodiment, the moiety is selected so as to maintain an alpha-helical coil structure or so as to facilitate a conformation compatible with formation of a homotrimer or heterologous six helix bundle.

In embodiments, the invention provides methods comprising contacting a host organism with an effective amount of a composition such as a peptide of the invention. In particular embodiments, said effective amount reduces or controls the ability of a SARS coronavirus or other coronavirus to infect, fuse, spread or proliferate in a host organism or cell, tissue, or organ thereof.

In an embodiment, a peptide or a nucleic acid molecule of the invention is isolated or purified as would be understood in the art. In an embodiment of such a peptide molecule where a particular corresponding nucleic acid molecule is shown as capable of encoding such peptide molecule, it is recognized that the peptide molecule can be generated using a different nucleic acid molecule as known in the art.

In an embodiment, the invention provides a peptide fragment of a peptide of the invention, wherein the peptide fragment comprises at least about 14 contiguous amino acid residues. In an embodiment, the invention provides a nucleic acid fragment of a nucleic acid molecule of the invention, wherein the nucleic acid fragment comprises at least about 42 contiguous nucleic acid residues.

Table of Sequence Listing Information.

| | | |
|---|---|---|
| SEQ | | |
| ID | | |
| NO: | Brief Description | Type |

| | | |
|---|---|---|
| 1 | SARS coronavirus (Urbani | DNA/RNA |
| | strain; Genbank | |
| | Accession No. AY278741) | |
| | S protein | |
| 2 | SARS coronavirus S | PRT |
| | protein | |
| 3 | SARS coronavirus S | DNA/RNA |
| | protein; HR-N | |
| 4 | HR-N, 882-1011 | PRT |
| 5 | HR-N1 (882-973) | DNA/RNA |
| 6 | automatic translation | PRT |
| 7 | HR-N2 (916-973) | DNA/RNA |
| 8 | automatic translation | PRT |
| 9 | HR-N3 (927-973) | DNA/RNA |
| 10 | automatic translation | PRT |
| 11 | HR-N4 (974-1011) | DNA/RNA |
| 12 | automatic translation | PRT |
| 13 | HR-N5 (882-916) | DNA/RNA |
| 14 | automatic translation | PRT |
| 15 | HR-N6 (888-922) | DNA/RNA |
| 16 | automatic translation | PRT |
| 17 | HR-N7 (895-929) | DNA/RNA |
| 18 | automatic translation | PRT |
| 19 | HR-N8 (902-936) | DNA/RNA |
| 20 | automatic translation | PRT |
| 21 | HR-N9 (909-943) | DNA/RNA |
| 22 | automatic translation | PRT |
| 23 | HR-N10 (916-950) | DNA/RNA |
| 24 | automatic translation | PRT |
| 25 | HR-N11 (923-957) | DNA/RNA |
| 26 | automatic translation | PRT |
| 27 | HR-N12 (931-965) | DNA/RNA |
| 28 | automatic translation | PRT |
| 29 | HR-N13 (938-972) | DNA/RNA |
| 30 | automatic translation | PRT |
| 31 | HR-N14 (945-979) | DNA/RNA |
| 32 | automatic translation | PRT |
| 33 | HR-N15 (952-986) | DNA/RNA |
| 34 | automatic translation | PRT |
| 35 | HR-N16 (959-993) | DNA/RNA |
| 36 | automatic translation | PRT |
| 37 | HR-N17 (966-1000) | DNA/RNA |
| 38 | automatic translation | PRT |
| 39 | HR-C1 (1147-1185) | DNA/RNA |
| 40 | automatic translation | PRT |
| 41 | HR-C2 (1165-1185) | DNA/RNA |
| 42 | automatic translation | PRT |
| 43 | HR-C3 (1158-1185) | DNA/RNA |
| 44 | automatic translation | PRT |
| 45 | HR-C4 (1151-1185) | DNA/RNA |
| 46 | automatic translation | PRT |
| 47 | HR-N10a extended | PRT |
| | (902-950) | |
| 48 | HR-C4a extended | PRT |
| | (1150-1185) | |
| 49 | SARS Urbani S protein, | PRT |
| | LOCUS AAP13441 | |
| 50 | HR-N10 (916-950) | PRT |
| 51 | HR-N10 analog; | PRT |
| | ala, lys, arg | |
| 52 | HR-N10 analog; Aib | PRT |
| 53 | HR-N10 analog; Dxg | PRT |
| 54 | HR-N10 analog; | PRT |
| | i, i + 4 bridge | |
| 55 | HR-N10 analog; | PRT |
| | i, i + 4 bridges | |
| 56 | HR-N10 analog; | PRT |
| | i, i + 7 bridges | |
| 57 | HR-N10 analog; ile and | PRT |
| | leu in hydrophobic core | |
| 58 | HR-C4 (1151-1185) | PRT |
| 59 | HR-C4 analog; ala, lys, | PRT |
| | arg | |
| 60 | HR-C4 analog; Aib | PRT |
| 61 | HR-C4 analog; Dxg | PRT |
| 62 | HR-C4 analog; i, | PRT |
| | i + 4 bridge | |
| 63 | HR-C4 analog; i, | PRT |
| | i + 4 bridges | |
| 64 | HR-C4 analog; i, | PRT |
| | i + 7 bridges | |
| 65 | HR-C4 analog; ile in | PRT |
| | hydrophobic core | |
| 66 | HR-C1 (1147-1185) | PRT |
| 67 | HR-C4a analog 1 | PRT |
| 68 | HR-C4a analog 2 | PRT |
| 69 | HR-C4a analog 3 | PRT |
| 70 | HR-C4a analog 4 | PRT |
| 71 | HR-C4a analog 5 | PRT |
| 72 | HR-C4a analog 6 | PRT |
| 73 | HR-C4a analog 7 | PRT |
| 74 | HR-C4a analog 8 | PRT |
| 75 | HR-N10 heptad 1 | PRT |
| 76 | HR-N10 heptad 2 | PRT |
| 77 | HR-N10 heptad 3 | PRT |
| 78 | HR-N10 heptad 4 | PRT |
| 79 | HR-N10 heptad 5 | PRT |
| 80 | HR-N10 heptads 1,2 | PRT |
| 81 | HR-N10 heptads 2,3 | PRT |
| 82 | HR-N10 heptads 3,4 | PRT |
| 83 | HR-N10 heptads 4,5 | PRT |
| 84 | HR-N10 heptads 1,2,3 | PRT |
| 85 | HR-N10 heptads 2,3,4 | PRT |
| 86 | HR-N10 heptads 3,4,5 | PRT |
| 87 | HR-N10 heptads 1,2,3,4 | PRT |
| 88 | HR-N10 heptads 2,3,4,5 | PRT |
| 89 | HR-C4 heptad 1 | PRT |
| 90 | HR-C4 heptad 2 | PRT |
| 91 | HR-C4 heptad 3 | PRT |
| 92 | HR-C4 heptad 4 | PRT |
| 93 | HR-C4 heptad 5 | PRT |
| 94 | HR-C4 heptads 1,2 | PRT |
| 95 | HR-C4 heptads 2,3 | PRT |
| 96 | HR-C4 heptads 3,4 | PRT |
| 97 | HR-C4 heptads 4,5 | PRT |
| 98 | HR-C4 heptads 1,2,3 | PRT |
| 99 | HR-C4 heptads 2,3,4 | PRT |
| 100 | HR-C4 heptads 3,4,5 | PRT |
| 101 | HR-C4 heptads 1,2,3,4 | PRT |
| 102 | HR-C4 heptads 2,3,4,5 | PRT |
| 103 | PCR primer | DNA |
| 104 | PCR primer | DNA |
| 105 | PCR primer | DNA |
| 106 | PCR primer | DNA |

It is recognized that regardless of the ultimate correctness of any mechanistic explanation or hypothesis, an embodiment of the invention can nonetheless be operative and useful.

### BRIEF DESCRIPTION OF THE FIGURES

FIG. 1 illustrates coiled-coil prediction analysis of the SARS-COV S glycoprotein.

FIG. 2 illustrates sequence information of HR-N and HR-C regions of SARS coronavirus S protein.

FIG. 3 illustrates circular dichroism spectra and temperature denaturation profiles.

FIG. 4 illustrates equilibrium ultracentrifugation analysis of the HR-N and HR-C peptides.

FIG. 5 illustrates interaction between HR-N and HR-C peptides including circular dichroism spectra and temperature denaturation profiles.

FIG. 6 illustrates HPLC analysis of the HR-N10/HR-C1 complex.

FIG. 7 illustrates gel electrophoresis data.

FIG. 8 illustrates sedimentation equilibrium analysis.

FIG. 9 illustrates helix orientation in the HR-N/HR-C complex.

FIG. 10 shows a schematic representation of the three different states of the coronavirus S fusion protein during viral entry.

FIG. 11 illustrates variations of HR-N and HR-C peptide sequences.

FIG. 12 illustrates covalently constrained HR-N and HR-C peptides.

FIG. 13 illustrates modulation of the “a” and “d” residue positions.

FIG. 14 illustrates modification of molecules with PEG.

FIG. 15 illustrates peptide conjugation to a carrier.

FIG. 16 illustrates conjugation of a trimer structure to a carrier and conjugation of a 6-helix structure to a carrier.

FIG. 17 illustrates helix template immunogens and preparation methods.

FIG. 18 illustrates HR-N peptide sequences, HR-N1 to HR-N17.

FIG. 19 illustrates HR-C peptide sequences, HR-C1 to HR-C4.

FIG. 20 illustrates a nucleotide sequence for a SARS HR-N region.

FIG. 21 illustrates a SARS S protein nucleotide sequence.

FIG. 22 illustrates analogs of HR-C peptides.

### DETAILED DESCRIPTION OF THE INVENTION

The invention may be further understood by the following non-limiting examples.

### EXAMPLE 1

### Structural studies of SARS-Coronavirus Spike S Fusion Protein and Peptides

While not wishing to be bound by any particular theory, a possible explanation of events or mechanisms is as follows.

The spike (S) glycoprotein of coronaviruses mediates viral entry into host cells. It is a type 1 viral fusion protein which characteristically contains two heptad repeat regions, denoted HR-N and HR-C, that form coiled-coil structures within the ectodomain of the protein. The two heptad repeat regions can undergo a conformational change from their native state to a 6-helix bundle (trimer of dimers) which is involved in mediating fusion of viral and host cell membranes.

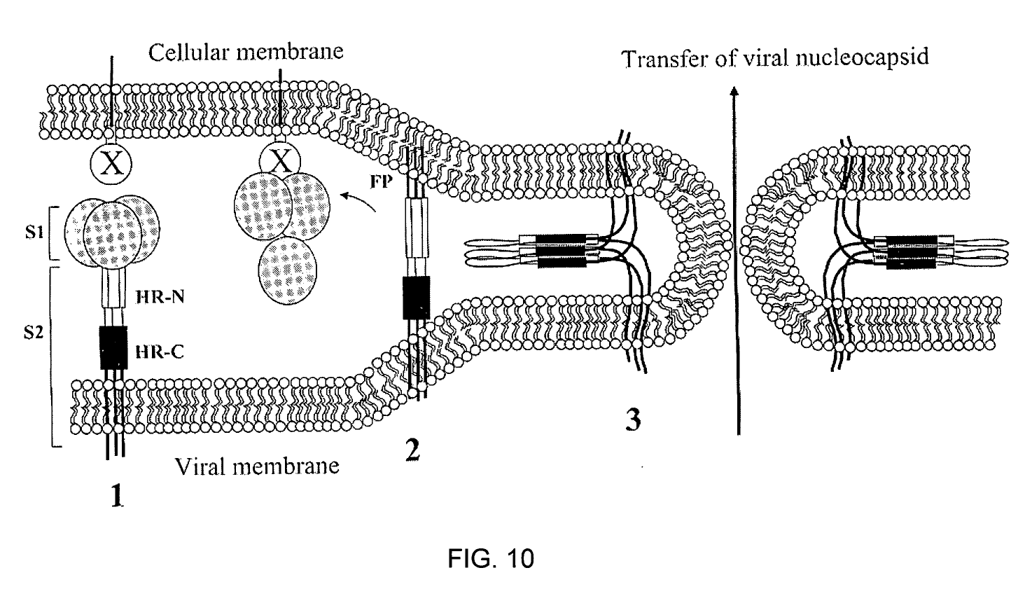

FIG. 10 is a schematic representation of the three different states of the coronavirus S fusion protein during viral entry. State 1 (native), the S protein is denoted S1 and S2 for its N- and C-terminal domains; state 2 (intermediate state), the N-terminal (S1) domain is dissociated (shedding) to expose the fusion peptide (FP) region, and state 3 (collapsed 6-helix bundle or fusion active state), the collapsed S2 domain draws the viral and cellular membranes together causing fusion and release of the viral nucleocapsid into the host cell. HR-N and HR-C denote coiled coils at the N terminus and C terminus of the S2 domain. An “X” within a circle denotes the host cell surface receptor.

It is thought that the binding of S protein of murine coronavirus MHV to a specific soluble or cell surface glycoprotein receptor induces global changes in the conformation of S protein that converts S protein from a native pre-fusogenic state to a fusion active state. In the case of the MHV-JHM strain, interaction with the host cell receptor CEACAM1 causes the S1 domain to readily dissociate from the S1/S2 complex, resulting in display of a new hydrophobic surface area that allows the virions to bind to liposomes and exposes previously hidden trypsin cleavage sites in S2 (21-23). This receptor-induced conformational change is believed to facilitate the release of a hydrophobic fusion peptide from the interior of S2 and position it to interact with the host cell membrane.

The fusion-active state of type 1 viral fusion proteins resolves itself to a fusogenic or post-fusion state (24). This change is thought to arise from an association between the two coiled-coil regions within the S2 subunit; in which the N-terminal HR region from three S proteins form a parallel triple stranded coiled-coil and the interhelical polypeptide chain loops around to reverse the polypeptide chain direction, positioning the HR-C region antiparallel to HR-N.

The positions of a heptad repeat unit are denoted as (abcdefg)ₙ, where n is a repeat number. The residues at the a and d positions (of the heptad repeat positions denoted (abcdefg)ₙ) of HR-C then pack into grooves formed by the residues at the a, d, e and g positions of the HR-N core coiled-coil to complete the 6-stranded alpha-helical bundle structure also termed “trimer of hairpins” or “trimer of dimers”. This structure ultimately draws the viral and cellular membranes close together, destabilizing the lipid bilayers which surround the virus and target cell, causing fusion and release of the viral nucleocapsid into the host cell.

Whether or not the above possible explanations or any depiction of a general membrane fusion mechanism (25-36) is accurate for the SARS virus and other coronaviruses, compositions and methods of the present invention can operate effectively, for example to modify or prevent initial infectivity and the spread of infection.

We describe the biophysical analysis of two heptad repeat regions within the severe acute respiratory syndrome (SARS) coronavirus S protein. Our results show that in isolation, the HR-N region forms a stable alpha-helical coiled-coil which associates in a tetrameric state. The HR-C region in isolation formed a weakly stable trimeric coiled-coil. Thus we demonstrate that the HR-N and HR-C regions of the SARS-CoV S glycoprotein can independently form alpha-helical coiled-coil structures.

When mixed together, the two peptide regions (HR-N and HR-C) associated to form a stable alpha-helical 6-stranded structure (trimer of heterodimers). Systematic peptide mapping showed that a site of interaction between the HR-N and HR-C regions is between residues 916 to 950 of the HR-N domain and residues 1151 to 1185 of the HR-C domain. Additionally, interchain disulfide-bridge experiments showed that the relative orientation of the HR-N and HR-C helices in the complex was antiparallel.

### FIGURE LEGENDS

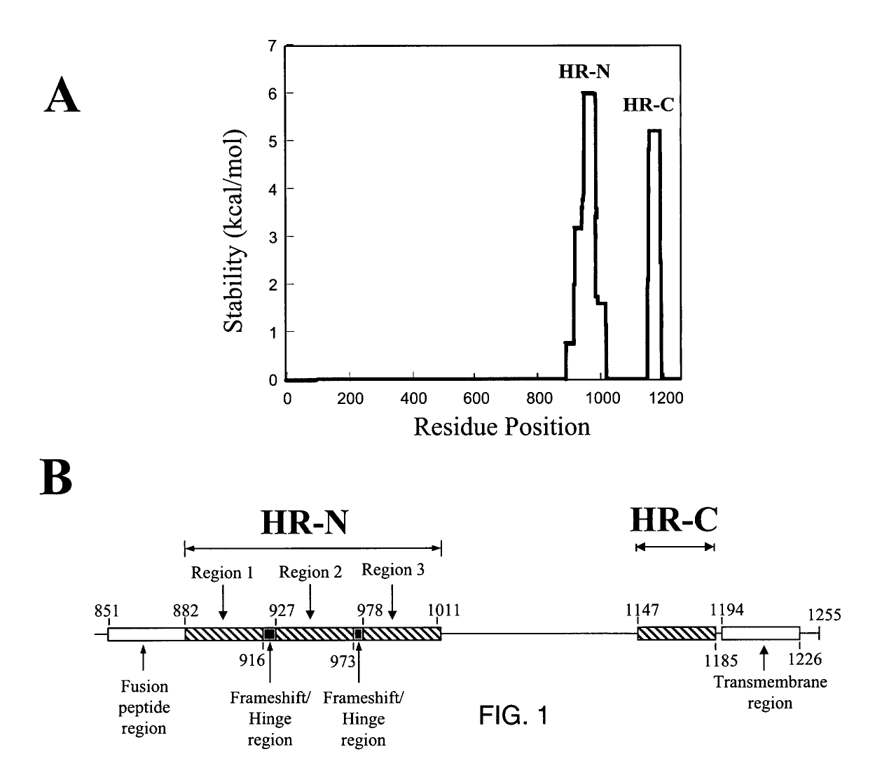

FIG. 1. Coiled-coil prediction analysis of the SARS-CoV S glycoprotein. (A) A plot of the predicted stability versus sequence position for the SARS-CoV S protein using the coiled-coil prediction algorithm STABLECOIL (53) with a 35 residue window width. The two regions of interest to this study are denoted HR-N (for N-terminal heptad repeat) and HR-C (for C-terminal heptad repeat). (B) A diagram representation of the relative locations of the predicted coiled-coils HR-N and HR-C within the S protein of the SARS-COV. The location of the predicted transmembrane spanning region, and fusion peptide region are also shown. HR-N can be divided into three sub-regions (regions 1, 2 and 3) based on the assignment of the a and d positions. Changes in the a and d register are denoted as a frameshift/hinge region.

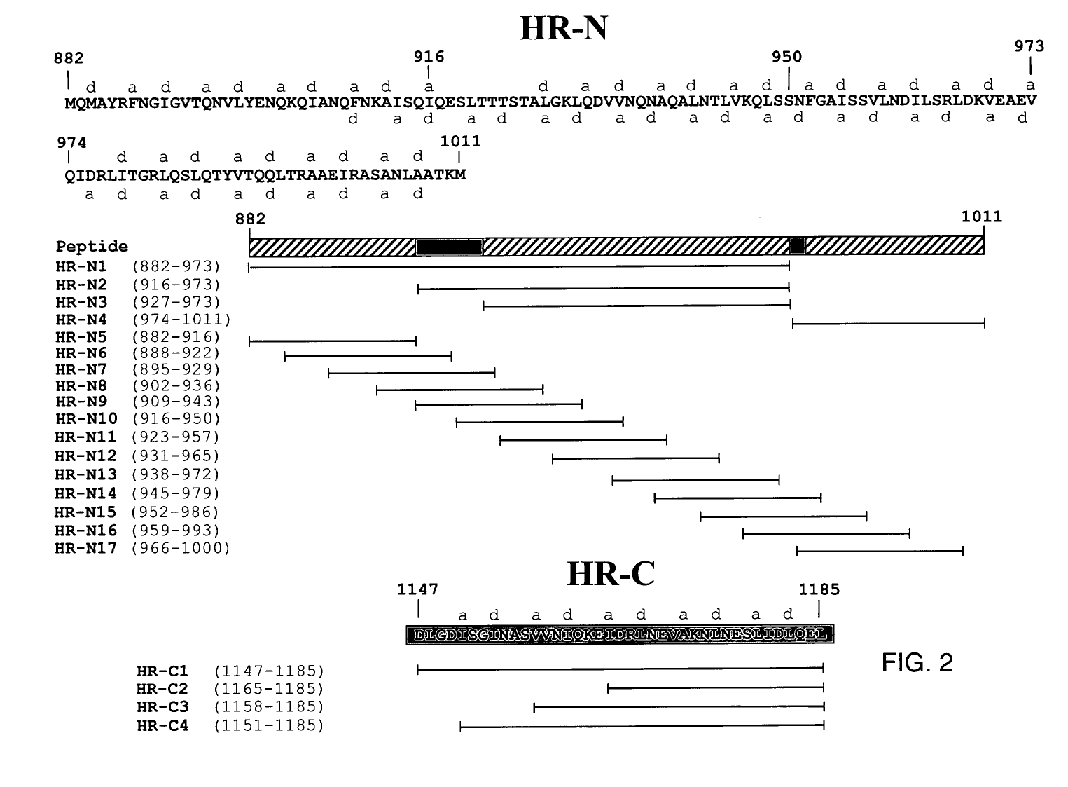

FIG. 2. (Top); Sequence of the predicted HR-N region from residues 882 to 1011 of the SARS-CoV S protein. The a and d positions of the strongest predicted coiled-coil heptad repeats (abcdefg)ₙ are shown above the sequence. The a and d positions of the alternate weaker scoring heptad repeat is shown below the sequence. (Middle); The names and sequence regions (denoted in brackets) of the HR-N peptides used in this study. The rectangle denotes the HR-N sequence from residue 882 to 1011. The hashed areas denote regions 1, 2 and 3 (FIG. 1). The bars underneath the HR-N domain indicate the locations of the peptides within the HR-N region used in this study. (Bottom); The sequence of the predicted HR-C domain from residues 1147 to 1185. The a and d positions are shown above the sequence. The names, sequences and locations (bars) of the HR-C peptides used in this study are shown below the HR-C domain.

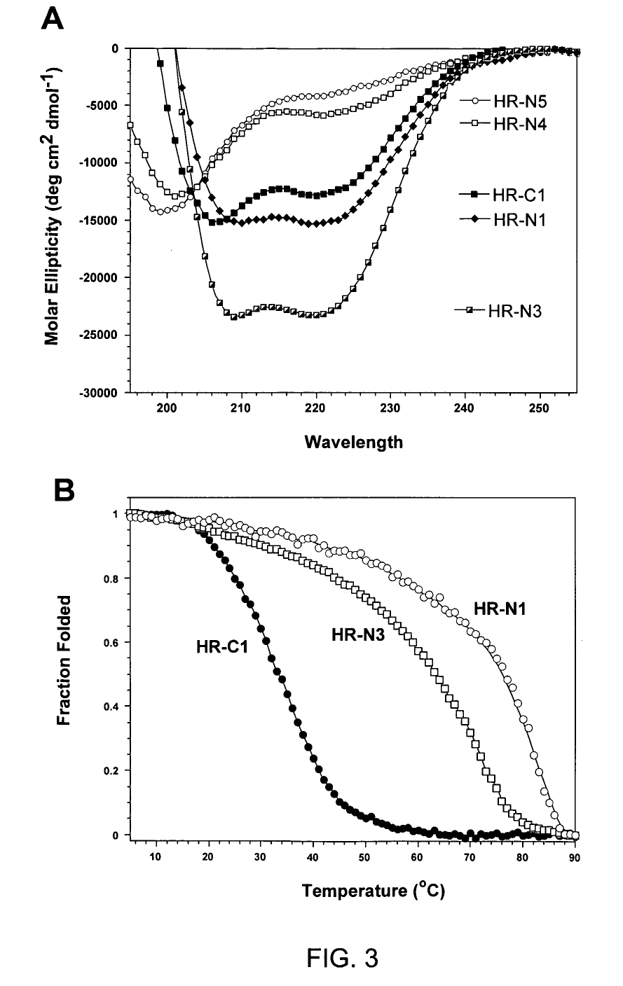

FIG. 3. (A) Circular dichroism (CD) spectra of peptides corresponding to the HR-N and HR-C of the SARS-COV S protein. Spectra were recorded in a 0.1 M KCl, 0.05 M PO₄, pH 7 buffer. Peptide concentrations were 100 μM for HR-N3 and HR-C1, and 15 μM for HR-N1, HR-N4 and HR-N5. (B) Temperature denaturation profiles of the HR-N and HR-C peptides monitored by CD at 222 nm in a 0.1 M KCl, 0.05 M PO₄, pH 7 buffer. Concentrations were 15 μM for HR-N1 and 100 μM for HR-N3 and HR-C1 peptides. The fraction folded (f_{f}) of each peptide was calculated as f_{f}([θ]−[θ]ᵤ)/([θ]ₙ−[θ]ᵤ), where [θ], theta, is the observed mean residue ellipticity at 222 nm at any particular temperature and [θ]ₙ and [θ]ᵤ are the mean residue ellipticities at 222 nm of the native folded state at 4° C. and unfolded states, respectively.

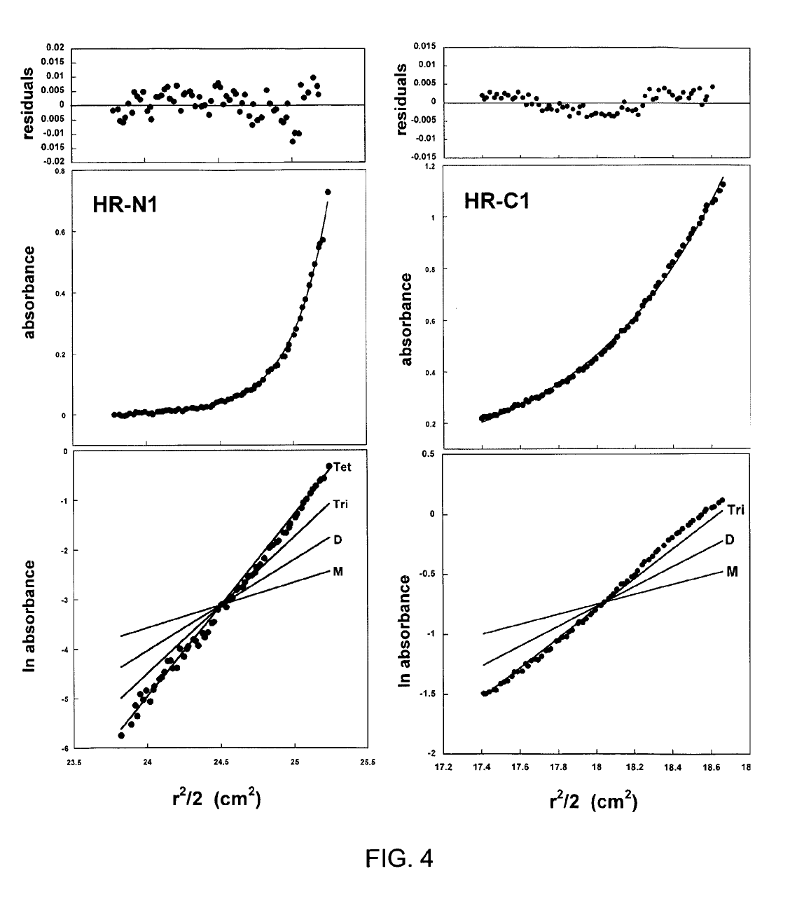

FIG. 4. Equilibrium ultracentrifugation analysis of the HR-N and HR-C peptides. (Left-side, middle;) a plot absorbance versus radial distance squared divided by 2 for HR-N1 at 22° C. and 26,000 rpm in a 0.1 M KCl, 0.05 M PO₄, pH 6 buffer. Protein concentration was 15 μM. (Left-side, bottom); a plot of the natural logarithm of the absorbance versus radial distance squared divided by 2 for the same data. The theoretical lines for a single species monomer (M), dimer (D), trimer (Tri) and tetramer (Tet) are shown for comparison. The data best fit a single species model with tetrameric molecular weight. (Right-side, middle); a plot of absorbance versus distance squared divided by 2 of the HR-C1 peptide at 22° C. and 30,000 rpm in a 0.1 M KCl, 0.05 M PO₄, pH 7 buffer. Peptide concentration was 250 μM. (Right-side, bottom); a plot of the natural logarithm of the absorbance versus radial distance squared divided by 2 for the same data. The theoretical lines for monomer (M), dimer (D) and trimer (Tri) are shown. The data was fit best to a monomer to trimer associating model in equilibrium. Residuals from both fits are shown above.

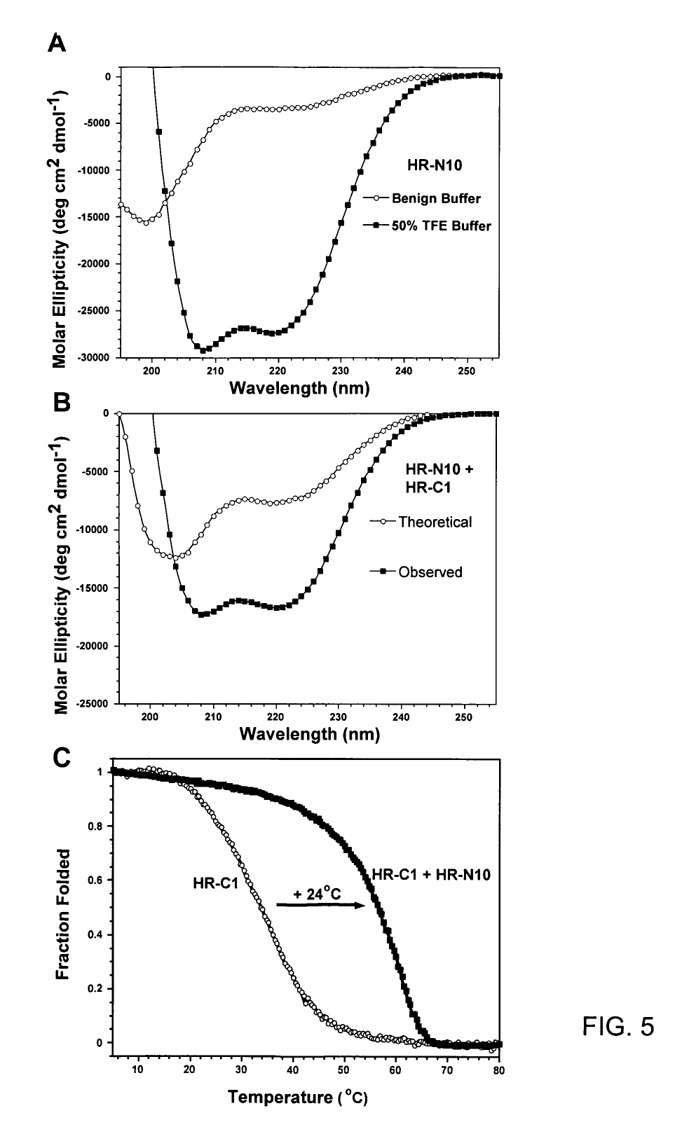

FIG. 5. Interaction between HR-N and HR-C peptides. (A) Circular dichroism spectra of HR-N10 peptide alone. Spectra were recorded at 25° C. in a 0.1 M KCl, 0.05 M PO₄, pH 7 buffer. For the spectrum containing 50% trifluoroethanol (TFE), the above buffer was diluted 1:1 (v/v) with TFE. (B) CD spectrum of a 1:1 molar complex between HR-N10 and HR-C1 peptides at 25° C. in a 0.1 M KCl, 0.05 M PO₄, pH 7 buffer. Peptide concentrations were 80 μM. The theoretical spectrum for two non-interacting peptides is shown for comparison. This spectrum is generated by adding the individual peptide spectra at the same concentrations. (C) Temperature denaturation profiles of HR-C1 alone and a 1:1 molar HR-C1 with HR-N10 complex monitored by CD at 222 nm in a 0.1 M KCl, 0.05 M PO₄, pH 7 buffer. Concentrations were 100 μM. Fraction folded was calculated as described in FIG. 2 legend.

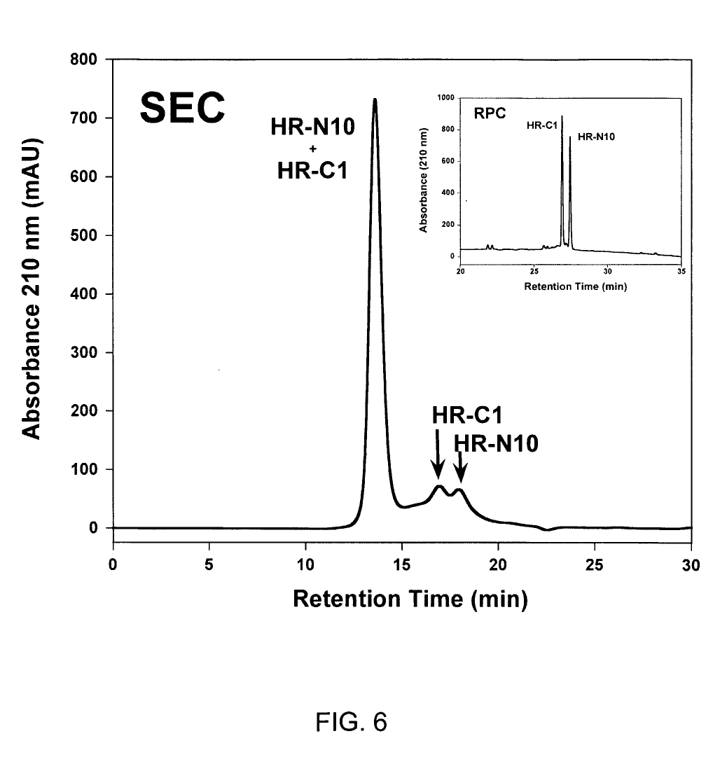

FIG. 6. HPLC analysis of the HR-N10/HR-C1 complex. HR-N10 (2 nmol) and HR-C1 (2 nmol) were pre-incubated together for 30 min in 10 μl running buffer and then applied to a Superdex™ 75 SEC column equilibrated in a buffer with 0.1 M KCl, 0.05 M PO₄, pH 7 and a flow rate of 0.7 ml/min. The absorbance peak at 13 min corresponding to the HR-N10/HR-C1 complex was collected and subsequently analyzed by reversed-phase chromatography (inset) on an analytical C8 Zorbax column employing a linear AB gradient of 2% B /min where A=0.05% aqueous TFA and B is 0.05% TFA/acetonitrile. Each absorbance peak is labeled accordingly.

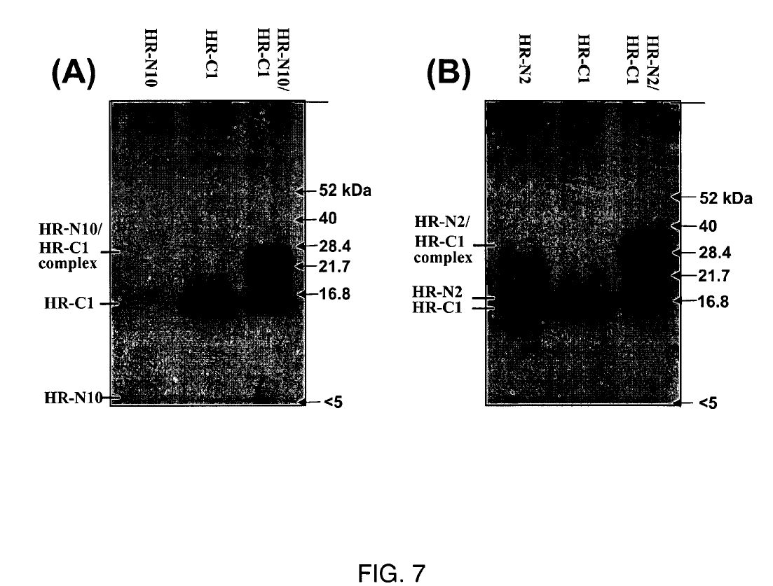

FIG. 7. Molecular mass of the complex formed between the HR-N and HR-C regions as determined by gel electrophoresis. (A) HR-N10 and HR-C1 peptides on their own or as pre-incubated equimolar (200 μM of each peptide) mixture were subjected to Tris SDS-15% PAGE. Samples were incubated for 30 min in 0.1 M KCl, 0.05 M PO₄, pH 7 buffer and the diluted 1:1 (v/v) with 2×Laemmli sample buffer at room temperature and loaded into the gel. The positions of HR-N10, HR-C1 and HR-N10/HR-C1 complex are indicated on the left side of the gel, while the positions of the molecule mass markers are indicated on the right side of the gels with arrows. The absence of the HR-N10 band was a result of the peptide eluting from the gel during the staining and destaining steps. (B) HR-N2 and HR-C1 peptides treated in a manner similar to that described in (A). The lanes of the gel, peptide locations and molecular weight markers are labeled accordingly.

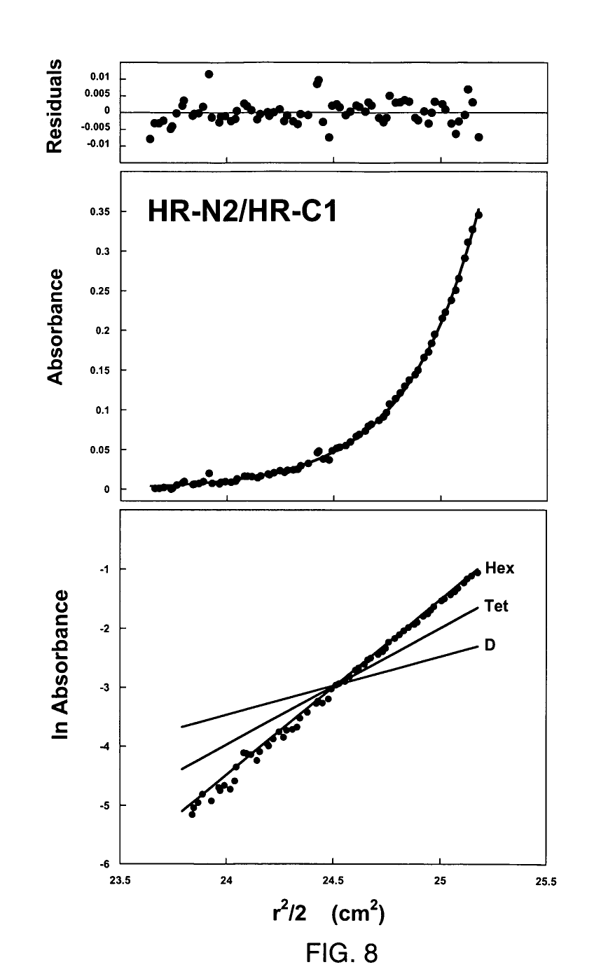

FIG. 8. Molecular mass of the complex formed between HR-N2 and HR-C1 peptides as determined by sedimentation equilibrium analysis at 22° C. and 30,000 rpm. The middle panel (absorbance versus radial distance squared divided by 2) shows the best fit curve for a single species model indicating a molecular mass of 30,500 Da. The theoretical mass for a 3:3 mole ratio HR-N2/HR-C1 hexamer is 31,692 Da. The upper panel shows the residuals from the curve fit, and the lower panel shows the In (natural logarithm) absorbance vs. r²/2 of the data compared to theoretical 1:1 dimer (D), 2:2 tetramer (Tet) and 3:3 hexamer (Hex).

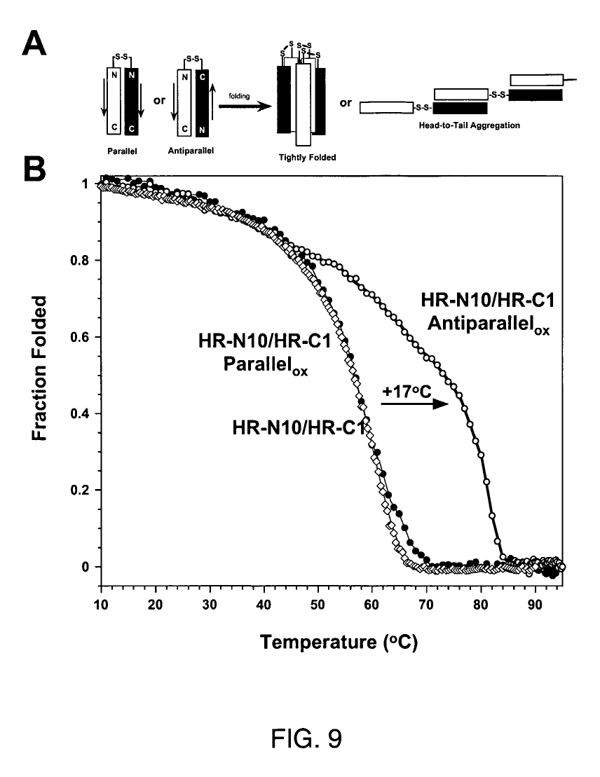

FIG. 9. Helix orientation in the HR-N/HR-C complex. (A) A diagram representation of the use of selective disulfide bridge formation to form parallel and anti-parallel hetero-stranded polypeptides, and the effect the relative helix orientations could have on the final folded state of the complex. The HR-N helix is represented as a white rectangle and corresponds to residues 916 to 950. The HR-C helix is represented as a black rectangle and corresponds to residues 1147 to 1185. The disulfide bridge between two cysteine residue sidechains is denoted by S-S. (B) Temperature denaturation profiles of parallel and anti-parallel disulfide bridged HR-N10/HR-C1 complexes in 0.1 M KCl, 0.05 M PO₄ pH 7 buffer monitored by CD at 222 nm. Peptide concentrations were 15 μM. Fraction folded was calculated as described in FIG. 2 legend. The temperature melting profile of the non disulfide bridged HR-N10/HR-C1 peptide is shown for comparison.

### METHODS

Peptide synthesis. The HR-N and HR-C peptides of SARS CoV S glycoprotein were prepared by solid-phase synthesis methodology using 4-benzylhydrylamine hydrochloride (MBHA) resin with conventional N-t-butyloxycarbonyl (t-Boc) chemistry as described by Tripet et. al., (47). Peptides were N-terminally acetylated, cleaved from the resin and purified by reversed-phase high performance liquid chromatography (RP-HPLC) to homogeneity and characterized by amino acid analysis and electrospray mass spectroscopy.

Plasmid construction. Production of the peptide corresponding to SARS S protein amino acids 882-973 (HR-N1) was done using directional sub-cloning and bacterial expression techniques. PCR fragments were prepared from the plasmid SARS S #18 (containing the entire S protein from SARS, Urbani strain (Genbank Accession No. AY278741)). Primers were designed to incorporate an Ndel restriction site upstream (GTACGTACGCATATGATGCAAATGGCATATAG GTTC) (SEQ ID NO:103) and an EcoRI site downstream (GCGMTTCCCTTTGTCGTCGTCGTC GCAGCCGCCTACCTCCGCCTCGACTTTATCMG) (SEQ ID NO:104) of the amplified fragment. The Ndel site contains an ATG start codon just upstream of the SARS sequence. The reverse primer was engineered to incorporate the amino acid sequence Gly-Gly-Cys-Asp-Asp-Asp-Asp-Lys to insert an enterokinase site (DDDDK) just downstream of the synthesized sequence, and just upstream of the EcoRI site. The nucleotide fragment corresponding to residues 882-973 was amplified by PCR and subcloned into the EcoRI/Ndel site of the pT7SH6 plasmid, in frame with the 6-His tag directly downstream of the EcoRI site to create the plasmid pT7SH6 SARS 882-973. pT7SH6 contains an oligonucleotide-encoded 6-His tag followed by two stop codons cloned into the EcoRI-BamHI sites of pT7.7 so that cloning into the EcoRI site creates a C-terminal protein fusion encoding NSHHHHHHXX.

Plasmid pT7.7 (based on the plasmids of Tabor and Richardson (48)) enables genes to be expressed by T7 RNA polymerase using the highly efficient translation signals of T7 gene 10 by cloning into and Ndel (CATATG) site where the ATG is the initiation codon. Plasmid SARS S #18 was created by cloning the full length SARS S protein into pcDNA3.1V5-His TA cloning vector (Invitrogen, Carlsbad, Calif.). The SARS S sequence was amplified using reverse-transcription from SARS genomic RNA (W. Bellini, CDC, Atlanta, Ga.), followed by PCR using primers F21488 (CACCATGTTTATTTTCTTATTATTT) (SEQ ID NO:105) and R25259 STOP (TTATGTGTAATGTMT TTGACACC) (SEQ ID NO:106). The reverse primer contains a stop codon to halt transcription before the V5-6-His tag in the cloning vector.

Bacterial Protein Expression and Purification. BL21 Al E. coli cells (Invitrogen, Carlsbad, Calif.) were transformed with pT7SH6 SARS 882-973. These cells were grown overnight in Luria Broth (LB) containing ampicillin (Sigma, St Louis, Mo.). The overnight culture was used to inoculate a flask of LB, containing ampicillin, and the cells were grown to an OD600 (optical density) of 0.7. The cells were induced by adding 10% L-Arabinose (Sigma) to a final concentration of 0.2%. Three hours later the cells were pelleted, resuspended in 1/10th volume of 20 mM tris/HCl pH 8.0 and protease inhibitors (Complete EDTA-free, Roche, Penzberg, Germany). The sample was frozen at −80° C. and thawed at 50° C. three times. A quantity of 1 mg DNAse (Promega, Madison, Wis.) was added to the sample and incubated at room temperature for 20 minutes. The sample was centrifuged at 14,000×g for 10 minutes. The soluble, supernatant medium, was purified with HiTrap nickel affinity columns (Pharmacia, Piscataway, N.J.) (49). The column was washed with 20 mM Tris/HCl pH 8.0, and bound proteins were eluted with a 0-500 mM gradient of Imidizole (Sigma) in 20 mM Tris/HCl pH 8.0. Fractions containing the SARS 882-973 (HR-N1) peptide were identified using immunoblot analysis with an anti-6-His polyclonal antibody (AbCam, Cambridge, Mass.). These fractions were pooled and placed over a reversed-phase chromatography column for purification.

Circular dichroism spectroscopy. Circular dichroism (CD) spectra were recoded on a Jasco J-810 spectropolarimeter (Jasco Inc., Easton, Md.). The CD wave scans were measured from 190 to 255 nm in benign buffer (0.1M KCl, 0.05M PO₄, pH 7). For samples containing trifluoroethanol (TFE), the above buffer was diluted 1:1 (v/v) with TFE. Temperature denaturation midpoints (T1/2) for the peptides were determined by following the change in molar ellipticity at 222 nm from 4° C. to 95° C. in a 1 mm path length cell and a temperature increase rate of 1° C./min. Ellipticity readings were normalized to the fraction of peptide folded (f_{f}) or the fraction of peptide unfolded (fᵤ) using the standard equations:

$${f}_{f} = \frac{( [ θ ] - {[ θ ]}_{u} )}{( {[ θ ]}_{n} - {[ θ ]}_{u} )} ;$$

$$and$$

$${f}_{u} = ( 1 - {f}_{f} ) ;$$

f_{f}=(] [symbol] ]−[ [symbol] ]ᵤ)/([ [symbol] ]ₙ−[ [symbol] ]ᵤ); and fᵤ=(1−f_{f}); where [ [symbol] ]ₙ and [ [symbol] ]ᵤ represent the elipticity values for the fully folded and fully unfolded species, respectively, and [ [symbol] ] is the observed ellipticity at 222 nm at any temperature.

Sedimentation analysis. Sedimentation equilibrium experiments were performed on the analytical ultracentrifuge (XLA from Beckman-Coulter) as described by Tripet et. al., (47). In general HR-N and HR-C peptides were dialyzed exhaustively against 50 mM K₂PO₄, 100 mM KCl, pH 7 buffer at 4° C. For full length HR-N1 peptide, HR-N1 was dialyzed against the low salt containing buffer 50 mM K₂PO₄, pH 6 for enhanced solubility. Three 100 μl aliquots of the sample were loaded into a 12 mm six-sector, charcoal-filled Epon cell and centrifugation proceeded for 48 hours with rotor speeds of 20,000, 26,000 and 30,000 rpm. The HR-N and HR-C peptides were each loaded at initial total peptide concentrations of 50, 100 and 250 μM whereas the full length HR-N1 peptide was loaded at 10, 20 and 50 μM. The partial specific volume of each peptide was calculated using the program SednTerp v. 1.08 (50). The density of the solvent was calculated to be 1.009 g/ml. The data were evaluated using a non-linear least squares curve-fitting algorithm contained in the WinNonLin analysis program v. 1.06 (51).

Gel electrophoresis. HR-N and HR-C region peptides singly or as an equimolar mixture (200 μM of each peptide) were dissolved in 100 mM KCl, 50 mM PO₄, pH 7 buffer and incubated at room temperature. Samples were then diluted with 1 volume of 2×Laemmli sample buffer and analyzed by SDS-PAGE in a 15% Tris/Glycine gel.

Size-exclusion and reversed-phase chromatography. HR-N and HR-C region peptides singly or as an equimolar mixture (200 μM of each peptide) were dissolved in 100 mM KCl, 50 mM PO₄, pH 7 and equilibrated at room temperature for 30 min. A 10 μl aliquot of the mixture was loaded onto a high-performance size-exclusion column, Superdex 75™ (1 cm×30 cm, Pharmacia, Uppsala, Sweden) equilibrated in a buffer with 50 mM PO₄, 100 mM KCl, pH 7 at a flow rate of 0.75 ml/min and ambient temperature. For peptides which formed stable heterostranded complexes, the complex peak was collected and analyzed by reversed-phase chromatography on an analytical C8 column (Zorbax 300SB-C8, 15 cm×4.6 mm l.D., 6.5 μM particle size, 300 Å pore size; Agilent Technologies). The peptides were eluted from the column by employing a linear AB gradient of 2% B/min, where eluent A is 0.05% aqueous TFA and eluent B is 0.05% TFA in acetonitrile at a flow rate of 1.0 ml/min at room temperature. To calculate the peptide ratio in the complex, the peak areas of each component were compared with peak areas of known standard solutions of each peptide.

Formation of parallel and anti-parallel disulfide-bridged hetero-two-stranded molecules. Preferential disulfide-bridge formation between the HR-N9 and HR-C1 regions was performed similar to that described by Semchuck et. al., (52). In brief, 10 mg of 2,2′-dithiopyridine (DTDP) was dissolved in 100 μl of dimethylformamide (DMF) with sonication. A 10 μl (3.4 μmoles) aliquot of this solution was added to a solution of the HR-N9 peptide (2 mg, 0.8 μmoles) dissolved in a 3:1 (v/v) acetic acid/H2O buffer and stirred for 6 h. The solution was then extracted 3× with 500 μl of ether and the aqueous layer was applied to a Sephadex G-25 desalting column conditioned with 50 mM NH₄Acetate, pH 5.5 running buffer. Fractions (1 ml) eluted from the column were collected and those with 220 nm absorbance were pooled. To one half of the pooled solution was added HR-C1 peptide with either a N-terminal CGG or a C-terminal GGC linker (1 mg dissolved in 1 ml of 50 mM NH₄Ac, pH 5.5. buffer) in 100 μl aliquots over 30 min. The reaction were then stirred for 1 h and the final complex purified by RP-HPLC and freeze dried.

### RESULTS

Sequence Analysis.

To identify the heptad repeat (HR) regions within the SARS-CoV S protein, we utilized the coiled-coil prediction algorithm STABLECOIL (53). FIG. 1A shows the graphical output of the analysis using a 35 residue window width. Two coiled-coil regions are predicted; the HR-N coiled-coil (residues 882 to 1011) and the HR-C coiled-coil (residues 1147 to 1185) with an interhelical domain of approximately 140 amino acid residues between HR-N and HR-C. The HR-C region is located adjacent to a predicted transmembrane region (residues 1194-1226), and the HR-N region is located C-terminal to a region of high sequence similarity to the fusion peptide (residues 851-882) (FIG. 1B). The coiled-coil prediction analysis also indicates that the HR-N region can be further sub-divided into three regions (1, 2 and 3) based on the assignment of the heptad a and d positions (data not shown). The region where the heptad registers change is denoted as a frameshift/hinge region (FIG. 1B). Also predicted is a second (lower scoring) heptad repeat register within HR-N region 2 which can run continuous with the heptad register observed in HR-N region 3 (shown below the sequence in FIG. 2, top). The presence of this alternate heptad register indicates that hydrophobic residues also occur in the interfacial e and g positions in this location. In contrast, the HR-C coiled-coil region shows only a single continuous heptad register (FIG. 2, bottom).

Circular Dichroism analysis of the HR-N and HR-C Regions.

To examine the structural characteristics of the HR-N and HR-C regions of the SARS CoV S protein, peptides (see FIG. 2) corresponding to various regions between residues 882 and 1185 were synthesized and analyzed by circular dichroism (CD) spectroscopy. FIG. 3 (panels A and B) show representative far UV CD spectra and thermal denaturation profiles of the HR-N1, HR-N3, HR-N4, HR-N5 and HR-C1 peptides. Analysis of the HR-N1 peptide, which represents almost the entire length of the predicted HR-N region showed limited solubility in 0.1 M KCl, 0.05 M PO₄, pH 7 buffer, thus the CD study could only be carried out at 15 μM. The CD profile of HR-N1 showed the characteristic α-helical spectrum with double minima at 208 and 222 nm. The molar ellipticity at 222 nm (−15,100°) corresponds to 40 helical residues out of the maximum 62 helical residues induced in 50% TFE for the molecule under the conditions analyzed. Temperature denaturation of HR-N1 showed the α-helical structure had high stability with 50% of its structure unfolded at 77° C. (Table 1).

### Table 1. Ellipticity and Stability of Synthetic Peptides

TABLE 1
Ellipticity and stability of synthetic peptides.

| | | | | No. Hellcal | No. Hellcal | | |
|---|---|---|---|---|---|---|---|
| Peptide | No. | [θ]₂₂₂b, | [θ]₂₂₂b, | Residues^{c}, | Residues^{c}, | Percent Helix^{d} | |
| Name^{a} | Residues | Benign | 50% TFE | Benign | 50% TFE | (Benign/TFE) | T_{1/2}, ° C.^{e} |
| HR-N1* | 92 | −15,100 | −25,800 | 40 | 62 | 65 | 77 |
| HR-N2 | 58 | −21,100 | −23,300 | 33 | 37 | 89 | 68 |
| HR-N3 | 47 | −24,600 | −21,500 | 32 | 28 | 100 | 63 |
| HR-N4⁺ | 38 | −6,100 | −31,300 | — | 34 | — | — |
| HR-N5⁺ | 35 | −4,300 | −24,200 | — | 24 | — | — |
| HR-N6 | 35 | −3,300 | −20,100 | — | 20 | — | — |
| HR-N7 | 35 | −2,800 | −24,200 | — | 24 | — | — |
| HR-N8 | 35 | −2,500 | −25,100 | — | 25 | — | — |
| HR-N9 | 35 | −2,800 | −26,800 | — | 27 | — | — |
| HR-N10 | 35 | −3,600 | −28,500 | — | 29 | — | — |
| HR-N11 | 35 | −3,200 | −25,200 | — | 25 | — | — |
| HR-N12 | 35 | −16,500 | −21,000 | 17 | 21 | 81 | 52 |
| HR-N13 | 35 | −8,700^{f} | −20,500 | — | 21 | — | — |
| HR-N14 | 35 | −7,600^{f} | −12,000 | — | 12 | — | — |
| HR-N15 | 35 | −6,200^{f} | −14,900 | — | 15 | — | — |
| HR-N16 | 35 | −4,800 | −19,000 | — | 19 | — | — |
| HR-N17 | 35 | −5,500 | −23,300 | — | 24 | — | — |
| HR-C1 | 39 | −13,600 | −23,800 | 15 | 26 | 58 | 33 |
| HR-C2 | 21 | −3,600 | −15,400 | — | 10 | — | — |
| HR-C3 | 28 | −5,800 | −19,200 | — | 16 | — | — |
| HR-C4 | 35 | −17,500 | −19,800 | 18 | 20 | 90 | 33 |

Table 1 Legend.
^{a}Name of each peptide studied. The sequence position of each peptide is shown in FIG. 1.
^{b}The mean residue molar ellipticities at 222 nm were measured at 22° C. in benign buffer (0.1 M KCl, 0.05 M K₂PO₄, pH 7). For samples containing TFE, the above buffer was diluted 1:1 (v/v) with TFE. Values are in units of (degrees · cm² · dmol⁻¹).
^{c}The number of α-helical residues was calculated based on the predicted molar ellipticity. The predicted molar ellipticity for a completely α-helical peptide was calculated using [theta]_{theoretical} = 40,000 × (1-4.6/N) where N is the number of residues in the polypeptide chain. The number of α-helical residues in benign or TFE conditions was calculated as the observed [θ] divided by the theoretical [θ].
^{d}the percent α-helix in benign medium at the concentration used was calculated as the number of helical residues in benign divided by the maximum inducible α-helical residues in TFE. Note that in oligomerizing α-helices the helical content is concentration dependent. Higher concentrations shift the monomer to oligomer equilibrium increasing the α-helical content based on a two state unfolding mechanism (random coil to coiled-coil)
^{e}T_{1/2} is the transition midpoint temperature at which there is a 50% decrease in molar ellipticity [θ]₂₂₂ compared to the fully folded peptide as determined by CD at 5° C. All peptides in which T_{1/2} was determined are boxed, for example, HR-N1, HR-N2, HR-N3, HR-N12, HR-C1, and HR-C4.
^{f}These peptides all showed a characteristic α-helical spectrum with minima at 208 and 222 nm even though the [θ]₂₂₂ was low.
*Results obtained at a peptide concentration of 15 μM instead of 100 μM used for the other determinations.
⁺Peptides were studied in 0.05 M K₂PO₄ buffer, pH 7 due to low solubility.

Mapping of the HR-N region further with smaller peptides showed that HR-N5 and HR-N4 (region 1 and 3) were also largely insoluble, and the limited amount that did dissolve (15 μM) exhibited random coil spectra (FIG. 3A). The peptides, however, could be induced into an alpha-helical conformation in the presence of 50% TFE, a helix inducing solvent, indicating they do have an intrinsic ability to be helical (Table 1). In contrast, the HR-N3 peptide corresponding to the central region of HR-N (region 2, FIG. 1) showed good solubility characteristics (>5 mg/ml) and exhibited a strong α-helical spectrum (FIG. 3A). The mean residue molar ellipticity value (−24,600°) indicated that HR-N3 was fully helical relative to the maximum inducible α-helix in TFE but only 68% helical relative to the theoretical value for a fully folded helical molecule of its 47-residue length (−36,000°). The thermal melting profile of this peptide showed that it had high stability similar to that of the longer length HR-N1 peptide (compare T_{1/2} values of 63° C. versus 77° C. for HR-N3 and HR-N1, respectively). An extension of HR-N3 produced a peptide HR-N2 of 58-residues. This peptide had a similar number of helical residues (Table 1) and a T_{1/2} of 68° C.

We analyzed the CD spectra of several overlapping 35-residue peptides shifted 1 heptad (7 residues) toward the C-terminus of the HR-N region beginning at HR-N5 and ending with HR-N17 (FIG. 2). All of these peptides showed mainly random-coil spectra with the exception of HR-N12, HR-13, HR-14 and HR-15 which displayed helical spectra profiles. Interestingly, these peptides corresponded to the same 47 residues of the sequence of HR-N3 which was also observed to be helical (FIG. 2, Table 1). It is also notable that the region in HR-N that was predicted to have the highest likelihood of forming a coiled-coil structure (FIG. 1A) corresponds with the HR-N3 sequence region identified to form a stable α-helical structure. Interestingly, the stability of peptides (as measured by their T_{1/2} values) for HR-N12 (T_{1/2} 52° C.), HR-N3 (T_{1/2} 63° C.), HR-N2 (T_{1/2} 68° C.) and HR-N1 (T_{1/2} 77° C.) increased with increasing chain length from 35, 47, 58 and 92 residues respectively (Table 1). The number of helical residues in benign medium for these peptides also increased with chain length from 17 to 40 residues.

The far UV CD spectrum of HR-C1 exhibited double minima at 208 and 222 nm (FIG. 3A) indicating α-helical structure. The mean residue molar ellipticity was −12,600 degrees at 100 μM and increased to −22,000 degrees at high concentrations (>500 μM) similar to the value observed in the presence of TFE. Temperature denaturation of HR-C1 showed the peptide has lower stability as 50% of the molecule could be unfolded at 33° C. (FIG. 3B, Table 1).

To see if we could define further the α-helical region within HR-C (39-residues), we analyzed three truncated peptides of HR-C1. CD analysis of HR-C2 and HR-C3 (21 and 28 residue length, respectively) displayed random coil spectra in benign buffer, whereas HR-C4 (35 residue length) showed α-helical structure with a [θ]₂₂₂ value of −17,500 degrees (Table 1). Additionally, the thermal melting profile of HR-C4 indicated the same temperature transition midpoint (33° C.) as full length HR-C1 (Table 1). Thus a possible minimal length required for folding of the HR-C region coiled-coil appears to be 35 residues corresponding to the sequence 1151 to 1185.

Oligomeric State of the HR Regions

Sedimentation equilibrium experiments were carried out to determine the oligomeric states of the HR-N and HR-C peptides. The HR-N1 peptide was studied at three different concentrations and three different rotor speeds. The data obtained fit well to a single-species model with a weight average molecular weight of 48,750 Da (FIG. 4, left). Surprisingly, this value corresponds well to that expected for a tetramer of HR-N1, based on the amino acid sequence (4×12,004=48,016 Da). The high quality of the single species fit at the different concentrations analyzed suggests that the HR-N1 peptide was exclusively tetrameric over the entire concentration range of the experiment. This result was surprising considering earlier analysis of fusion proteins and ectodomain cores of other viruses have been observed to be trimeric (54-59). Our results, however, were consistent with the sedimentation equilibrium analysis of the smaller truncated HR-N peptide, HR-N3 which also displayed α-helical structure. Sedimentation equilibrium data obtained for this peptide best fit to a single species model with weight average molecular weight of 19,000 Da, close to that expected for a tetramer (theoretical molecular weight 20,212 Da, 4×5,053 Da, data not shown).

In contrast, sedimentation equilibrium experiments of the HR-C1 peptide at three different concentrations and three speeds showed that the data was best fit globally to an associating monomer to trimer equilibrium model. FIG. 4 (right) shows a plot of the absorbance versus radial distance squared divided by 2 and the residuals of the fit for the HR-C1 peptide. At the highest concentration (250 μM) and highest speed (30,000 rpm) the HR-C1 peptide showed a weight average molecular weight of 13,900 Da, slightly greater that the theoretical mass for a trimer (13,007 Da).

Interaction between the HR-N and HR-C.

The HR-N and HR-C of the murine coronavirus MHV S glycoprotein self associate to form a 6-helix bundle structure typical of other type 1 viral fusion proteins (19). To identify the significant sequences within the SARS-CoV S glycoprotein, mixtures of the HR-N and HR-C peptides were screened by CD spectroscopy and size-exclusion chromatography (SEC). Initially, mixtures between the three HR-N peptides: HR-N5, HR-N3 and HR-N4 (which correspond to regions 1, 2 and 3 of HR-N, FIG. 1) with HR-C1 were screened. These peptide mixtures demonstrated no detectable interaction, i.e., the CD spectra were super-imposable with the theoretical spectra calculated for two non-interacting peptides. Additionally, the SEC chromatogram profiles were similar to those of the individual peptides alone. We next analyzed the HR-N peptides corresponding to the intervening frameshift/hinge regions (FIG. 1). No interactions were observed for peptides HR-N6, HR-N7 and HR-N8. In contrast, HR-N9 showed the induction of 3,500 degrees at 222 nm, HR-N10 showed the induction of 9,600 degrees, and shifting 7 residues (1 heptad) further toward the C-terminus (HR-N11) resulted in the absence of any observable interaction once again with HR-C1 (Table 2 and FIG. 5). Similar mapping results were also observed in the temperature denaturation profiles and SEC analysis of the mixtures. The mixture between HR-N9/HR-C1 showed a T_{1/2} of 36° C., a 3° C. rise relative to HR-C1 alone (T_{1/2} 33° C.), and the SEC chromatogram showed the formation of a higher molecular weight complex of about 30% of the observed area units. HR-N10/HR-C1 (T_{1/2} 57° C.) showed a 24° C. increase in T_{1/2} from HR-C1 (FIG. 5C) and the SEC chromatogram showed largely one single peak of higher molecular weight (FIG. 6). The mixture between HR-N11/HR-C1 showed no complex peak in the SEC chromatogram. It is worthy of mention that the CD signal at 222 nm for the strongest interacting peptides (HR-N10/HR-C1) only showed a molar ellipticity of −17,700, about 51% of a theoretical fully helical peptide of 35-residues in length despite the significant increase in stability observed (data not shown).

### Table 2. CD and SEC Analysis of HR-N/HR-C Peptide Mixtures.

TABLE 2
CD and SEC analysis of HR-N/HR-C peptide mixtures.

| | | | Change in | | |
|---|---|---|---|---|---|
| | [θ]₂₂₂b, | [θ]₂₂₂b, | Molar | | |
| Peptide Mixture^{a} | Observed | Predicted | Ellipticity^{c} | T_{1/2}, ° C.^{d} | SEC^{e} |
| HR-N2/HR-C1 | −17,900 | −17,000 | 900 | 74 | (++) |
| HR-N3/HR-C1 | −18,000 | −18,000 | 0 | | (−) |
| HR-N4/HR-C1* | −9,400 | −9,400 | 0 | | (−) |
| HR-N5/HR-C1* | −8,500 | −8,500 | 0 | | (−) |
| HR-N6/HR-C1 | −8,600 | −8,600 | 0 | | (−) |
| HR-N7/HR-C1 | −7,400 | −7,400 | 0 | | (−) |
| HR-N8/HR-C1 | −7,900 | −7,900 | 0 | | (−) |
| HR-N9/HR-C1 | −10,800 | −7,300 | 3,500 | 36 | (+) |
| HR-N10/HR-C1 | −17,700 | −8,100 | 9,600 | 57 | (++) |
| HR-N11/HR-C1 | −8,000 | −8,000 | 0 | | (−) |
| HR-N12/HR-C1 | −14,900 | −14,900 | 0 | | (−) |
| HR-N13/HR-C1 | −10,300 | −10,300 | 0 | | (−) |
| HR-N14/HR-C1 | −12,500 | −12,500 | 0 | | (−) |
| HR-N15/HR-C1 | −9,500 | −9,500 | 0 | | (−) |
| HR-N16/HR-C1 | −9,000 | −9,000 | 0 | | (−) |
| HR-N17/HR-C1 | −9,500 | −9,500 | 0 | | (−) |
| HR-N10/HR-C2 | −3,800 | −3,800 | 0 | | (−) |
| HR-N10/HR-C3 | −4,900 | −4,900 | 0 | | (−) |
| HR-N10/HR-C4 | −17,900 | −10,500 | 7,400 | 56 | (++) |
| HR-N10/HR-C1(antiparallel)ox | −19,200 | | | 76 | |
| HR-N10/HR-C1(antiparallel)ox | −13,300⁺ | | | 74⁺ | |
| HR-N10/HR-C1(parallel)ox | −12,200⁺ | | | 57⁺ | |

Table 2 Legend.
^{a}Name of the peptides in the mixture. For sequence regions see FIG. 1. Names followed by “ox” indicate peptide mixtures which have been covalently linked by a disulfide bridge.
^{b}The mean residue molar ellipticities at 222 nm were measured at 22° C. in benign buffer (0.1 M KCl, 0.05 M K₂PO₄, pH 7). Peptide complexes scanned in a 0.05 M K₂PO₄ buffer are indicated with an asterisk (*). Peptide complex concentration was 100 μM and 15 μM (indicated with a cross, +). The predicted molar ellipticity for two non-interacting peptides was calculated by summing the two individual spectra. Values are in units of(degrees · cm² · dmol⁻¹).
^{c}The difference in molar ellipcity from the observed and predicted values (column 2 - column 3; observed minus predicted).
^{d}T_{1/2} is the transition midpoint temperature at which there is a 50% decrease in molar ellipticity [θ]₂₂₂ compared to the fully folded peptide as determined by CD at 5°C. When no change in molar ellipticity (column 3) was observed, the t_{1/2} was not determined. Peptide complex concentration was 100 μM and 15 μM (indicated with a cross, +).
^{e}Summary of the analysis of the peptide complexes by size-exclusion chromatography (SEC). (−) denotes no complex formation; (+) denotes weak complex formation between the two peptides (about 30% of total peak arrea or less); (++) denotes strong complex formation between the two peptides (>95% of total peak area).

To confirm that the structure of the HR-N region is not a factor in binding to HR-C1, as HR-N10 was a random coil on its own (FIG. 5A), we extended the length of HR-N3 (residues 927 to 973) which exhibited an α-helical structure by 11 additional N-terminal residues (HR-N2) to contain the complete binding site of HR-N10. This peptide showed strong complex interactions with HR-C1 as assessed by SEC. Further, the temperature denaturation profile indicated the midpoint rose 6° C. from 68° C. for HR-N2 alone to 74° C. for the complex HR-N2/HR-C1 (Table 1 and Table 2). The denaturation profile was cooperative for this complex versus a biphasic transition profile observed for HR-N3 with HR-C1 which showed T_{1/2} values corresponding to the individual domain T_{1/2} values indicative of non-interacting helices. Thus HR-C1 forms a complex with the HR-N region whether the N-region is unfolded (HR-N10) or folded (HR-N2).

We also analyzed the truncated peptides of HR-C1 in an attempt to localize the binding site for HR-N10. HR-C2 and HR-C3 peptides of 21 and 28 residues showed no ability to interact the HR-N10 region as assessed by CD spectroscopy and SEC (Table 2). In contrast, the HR-C4 peptide of 35-residues which folded independently into an α-helical structure showed strong complex formation with the HR-N1 peptide observed by CD and SEC (Table 2). For example, the temperature denaturation profile of HR-N10/HR-C4 displayed a similar temperature midpoint and molar ellipticity observed for HR-N10/HR-C1 (compare 57° C. with 56° C. and −17,700 with −17,900, respectively). Thus the binding interaction between the HR-N and HR-C regions appears to be localized to residues 916 to 950 in the center of HR-N and 1151 to 1185 within the HR-C region (based on the N-terminal truncations).

HR-N and HR-C form a 3:3 Hexameric Complex.

To determine the ratio of the peptides within the HR-N10/HR-C1 complex, mixtures of HR-N10 and HR-C1 peptides at molar ratios of 1.5:1, 1:1 and 1:1.5, respectively, were pre-incubated for 30 min and then applied to a size exclusion column (FIG. 6). The earlier eluting complex was collected and applied to a reversed-phase C8 analytical HPLC column and eluted (FIG. 6 inset). Both HR-N10 and HR-C1 peptides were observed. Integration of the peak areas for each component and conversion of these values to mole amounts showed that the two peptides within the complex were present at a 1:1 molar ratio.

Next, we analyzed the molecular mass of the complex. Bosch et. al., (19) had shown for MHV-A59 that complexes of HR-1 and HR-2 are stable during SDS-PAGE as long as the samples are not heated before loading. Therefore, HR-C1, HR-N10 or HR-N2 peptides separately or as pre-incubated equimolar mixtures were subject to Tris SDS-15% PAGE (FIG. 7). The HR-N10 peptide, which CD spectroscopy had shown to be a random coil, migrated in the gel to a location corresponding to its molecular weight (about 3800 Da). In contrast, the HR-C1 peptide and HR-N10/HR-C1 complex migrated with higher molecular masses. The mobility of the HR-C1 peptide ran according to a molecular mass of about 13 kDa (just below the 16.8 kDa marker) indicating a trimeric state (theoretical mass of 13,008 Da), and the HR-N10/HR-C1 complex migrated according to a molecular mass of about 24 kDa corresponding to a hexameric (6 stranded) complex (theoretical mass of 24, 279 Da) (FIG. 7A).

Similar results were also seen for the HR-N2/HR-C1 peptides (FIG. 7B). The HR-N2 peptide migrated as a broad band between about 24 kDa and about 17 kDa with the most pronounced band at about 17 kDa, indicating a molecular mass between tetramer and trimer (theoretical masses 24,512 and 18,384 Da, respectively), the HR-C1 peptide migrated according to a mass of about 13 kDa (close to a trimeric mass) and the HR-N2/HR-C1 complex migrated according to a molecular mass of 29 kDa, close to the mass of a 3:3 hexamer (theoretical mass 31,692).

Sedimentation equilibrium analysis was carried out on the more stable HR-N2/HR-C1 complex. As shown in FIG. 8, the data was fit best to a single species model with molecular mass of 31,500 Da, close to the theoretical mass of a 3:3 mole ratio (6 stranded) complex between the HR-N2 and HR-C1 peptides (theoretical mass 31,692 Da).

Antiparallel Helix Orientation.

To investigate the orientation of the helices within the HR-N10/HR-C1 complex, we chose to form disulfide-bridged heterodimers between HR-N10 and HR-C1 in the parallel and antiparallel polypeptide chain orientation. The premise of this experiment was that the correct polypeptide chain orientation should form a tightly folded helical structure of high solubility and greater stability due to elimination of the concentration dependence between the two peptides, whereas the incorrect orientation would form head-to-tail complexes, which would aggregate and show little or no increase in stability (FIG. 9A). To do this, we added Cys-Gly-Gly residues to the N-terminus of HR-N10 and either the N- or C-terminus of HR-C1. The peptides were then selectively covalently linked using the thiol coupling agent DTDP (see methods).

Table 2 and FIG. 9 show helicity and stability of the two disulfide bridged complexes. The first complex in which the HR-N10 and HR-C1 peptides were linked in an anti-parallel orientation showed high solubility (>5 mg/ml or 1 mM) and a temperature transition midpoint of 76° C., an increase of 19° C. relative to the non-disulfide bridged complex analyzed at the same concentration (HR-N10/HR-C1 complex, T_{1/2} of 57° C., Table 2). In contrast, the complex between HR-N10 and HR-C1 disulfide-bridged in the parallel orientation was largely insoluble in the same buffer conditions (solubility of about 15 μM). The low solubility was completely dependent upon the disulfide-bridge link between the peptides, as addition of dithiothreitol to reduce the disulfide-bridge completely reversed the solubility characteristics and allowed the complex to be fully soluble at high concentrations once again. A temperature denaturation melting profile was obtained at 15 μM for the parallel complex and compared to the anti-parallel complex at the same concentration (FIG. 9B). The temperature transition midpoint of the parallel complex was 57° C., similar to that observed for the non-disulfide bridged complex, indicating the interactions between HR-N10 and HR-C1 are likely inter-molecular, whereas the anti-parallel complex showed a temperature transition midpoint of 74° C., indicating that it was 17° C. greater than the opposite orientation. It is noteworthy that a CD scan of the anti-parallel complex at 100 μM concentration only showed a molar ellipticity reading of −19,200 degrees at 222 nm (a fully folded α-helix of 35-residues is predicted to have a molar ellipticity of −34,700°). Thus the highly stable complex either has unfolded residues at the termini of the helices or when in a complex, one or the other helix exists partly in a non-helical form. Taken together, however, the results suggest the HR-N and HR-C regions associate in an anti-parallel manner in the hexameric complex state.

### DISCUSSION

Here we used CD spectroscopy, SEC and sedimentation equilibrium analysis to characterize predicted HR-N and HR-C regions in the ectodomain of the spike S glycoprotein of the SARS-COV. In isolation, the HR-N region folds as an independent α-helical coiled-coil structure of high stability, but with only about 40 helical residues of a possible 92 residues. Analysis of smaller peptides of HR-N showed that the helical content is largely localized to residues 927 to 973. The central portion of HR-N and the N- and C-termini were unfolded.

It is interesting that the N-terminal region closest to the predicted fusion peptide sequence (residues 851 to 882, FIG. 1, a region of high sequence similarity to the fusion peptides of other type-1 fusion proteins which is thought to insert into the target membrane during the fusion process) did not show any α-helical coiled-coil structure indicating this region does not spontaneously fold into a coiled-coil and therefore requires other conditions or parts of the molecule. This is similar to observations in influenza HA, Ebola virus GP2, human T-cell leukemia virus type 1 gp21 and RSV F fusion proteins. In the case of influenza HA, the N-terminus of the HA₂ coiled-coil at neutral pH exists in part as an extended conformation, but undergoes a conformational change to a more stable elongated coiled-coil only when HA encounters a low pH acid environment in a series of steps termed the spring loaded mechanism (60-63). We analyzed the HR-N1 peptide at lower pH (4.5) and did not observe any significant increase in helicity (data not shown). CoV S proteins are generally stable at acid pH, and MHV S can be triggered to its fusogenic state at pH 8, 37° C. (23).

Studies on the HR regions of the S protein of the mouse hepatitis virus strain A59 showed the α-helical content of the separate HR-N and HR-C peptides were calculated to be about 89%, and that the helical content of their equimolar mixture was calculated to be about 82% (19). Further, electron microscopy showed rod-like structures of about 14.5 nm, correlating well with the length predicted for an α-helix the size of HR-N (96 residues). Since the sequences between coiled-coil domains of the SARS-CoV Urbani strain analyzed here and MHV-A59 have a high degree of sequence identity, this difference was quite surprising. One possibility for the difference may be due to the buffer conditions in which the samples were analyzed. The MHV-A59 peptide was analyzed in H₂O versus physiological conditions used in this study (100 mM KCl, 50 mM PO₄, pH 7 buffer). Alternatively, the difference could be due to the low concentration at which we analyzed SARS HR-N1 (15 μM). The α-helical content of peptides that form coiled-coil structures are affected by concentration due to the monomer-oligomer equilibrium (two-state folding mechanism random coil to coiled-coil). We observed a strong α-helical coiled-coil in HR-N between residues 927 to 973. This region correlates with the protease resistant portion of the HR-N region of MHVS, residues 976 to 1040 (19), corresponding to residues 909 to 973 of the SARS-CoV S protein sequence.

Sedimentation equilibrium analysis showed that both the long and short folded HR-N region peptides associated as a tetramer; however, in SDS-PAGE gel electrophoresis the shorter HR-N region (HR-N2) displayed a smear between tetramer and trimer with the more dominant band displaying a trimeric molecular loss in the ability to interact with HR-C1. The HR-N10 region is a significant site of interaction with HR-C1.

Despite CD analysis of the HR-N10/HR-C1 complex showing a significant increase in thermal stability and α-helical induction, the complex did not adopt a fully helical structure (−17,700° molar ellipticity observed). Some loss can be attributed to end fraying and concentration dependence (66-68). However, when HR-N10 and HR-C1 were disulfide-bridged, removing the concentration dependence between these two peptides, the molecule only displayed −19,200 degrees molar ellipticity. One possible explanation may be that the interaction between HR-C and HR-N10 is not fully helical, but more akin to the binding interactions observed for HR-C to HR-N of the F protein of the paramyoxovirus SV5 (25). The structure of the fusion-active core region of the SV5 F protein shows that the HR-C (HR2) domain contains both an α-helix and an extended conformation that stretches out along the HR-N (HR2) region. Further characterization of the binding interactions for S of SARS-COV is resolved by high resolution structural studies.

Viral fusion proteins can adopt at least two different conformations (i.e., native and fusogenic states), and the change in conformation is thought to be required for fusion of the viral and cellular membranes during virus entry (24). The α-helical hetero-trimeric (6-helix) conformation of the complex of HR-N10 and HR-C1 peptides of SARS-COV S protein is a structure that can represent the core of the fusion competent state of the SARS-CoV S protein. Without wishing to be bound by any particular theory, a possible explanation is that the 6-helix form of the HR-N10 and HR-C1 peptides displays greater stability than the separate domains, and an energy release from conformational change to a more stable form may provide the energy to overcome the activation energy needed to bring the two lipid bilayers close together at the fusion site (60, 62, 63).

Also without wishing to be bound by any particular theory, a possible explanation of a fusion mechanism is as follows. In the native state, the two HR regions of the fusion protein ectodomain oligomerize to form homo-trimeric coiled-coil cores, which when stimulated by receptor interaction undergo a conformational change to an antiparallel 6-stranded α-helical structure (trimer of dimers) in the fusion active state to initiate the fusion of viral and cellular membranes. Our observation of the 6-helix bundle conformation in connection with SARS therefore leads to compositions such as peptides corresponding to the SARS-CoV S protein HR regions that can modify viral fusion. For example, a peptide composition can inhibit viral fusion for SARS and optionally other coronaviruses. A composition can comprise free HR-N or HR-C peptides, complexes of each peptide, or complexes of a peptide mixture. Such compositions can compete for or affect binding to the HR-N and HR-C coiled-coils in the native state, thereby blocking the conformational transition to the fusion-active form. For example, a homotrimer of HR-N10 can function to disrupt the ability of a SARS S protein to adopt or maintain a fusion competent conformation, thereby inhibiting infectivity or spread of a SARS coronavirus.

### EXAMPLE 2

### Preparation of Peptide Products

The peptides are prepared by synthetic chemistry methods. Methods known in the art such as those utilizing t-Boc/Benzyl or Fmoc/t-Boc chemistry are used. See, e.g., Solid phase peptide synthesis, John M Stewart and Janis D Young, Second edition, 1984, Pierce Chemical Company; Fmoc solid phase peptide synthesis, A practical approach, W. C. Chan and P. D. White, Editors, 2000, Oxford University Press.

The peptides are prepared using recombinant expression technology. For example, a desired gene sequence is used as an insert in connection with an expression vector and transformed into an appropriate host for expression.

Standard techniques for cloning, isolation, amplification and purification of DNA, RNA, and protein, for enzymatic reactions involving DNA ligase, DNA polymerase, restriction endonucleases and the like, and various separation techniques are those known and commonly employed by those skilled in the art. A number of standard techniques are described in Sambrook et al. (1989) Molecular Cloning, Second Edition, Cold Spring Harbor Laboratory, Plainview, N.Y.; Maniatis et al. (1982) Molecular Cloning, Cold Spring Harbor Laboratory, Plainview, N.Y.; Wu (ed.) (1993) Meth. Enzymol. 218, Part I; Wu (ed.) (1979) Meth Enzymol. 68; Wu et al. (eds.) (1983) Meth. Enzymol. 100 and 101; Grossman and Moldave (eds.) Meth. Enzymol. 65; Miller (ed.) (1972) Experiments in Molecular Genetics, Cold Spring Harbor Laboratory, Cold Spring Harbor, New York; Old and Primrose (1981) Principles of Gene Manipulation, University of California Press, Berkeley; Schleif and Wensink (1982) Practical Methods in Molecular Biology; Glover (ed.) (1985) DNA Cloning Vol. I and II, IRL Press, Oxford, UK; Hames and Higgins (eds.) (1985) Nucleic Acid Hybridization, IRL Press, Oxford, UK; and Setlow and Hollaender (1979) Genetic Engineering: Principles and Methods, Vols. 1-4, Plenum Press, New York.

Also see current editions in the series of Current Protocols titles (all generally published by John Wiley and Sons, New York), e.g. Current Protocols in Molecular Biology (edited by Frederick M. Ausubel et al., 1991-2004, New York: Greene Pub. Associates and Wiley-lnterscience: J. Wiley); Current Protocols in Cell Biology, Current Protocols in Cytometry, Current Protocols in Immunology (edited by John E. Coligan, et al., New York: John Wiley and Sons, 1994-1998), Current Protocols in Pharmacology, Current Protocols in Protein Science; and Current Protocols in Toxicology.

In one example, a recombinant peptide is prepared comprising a single peptide such as HR-N10 or HR-C4. In another example, a recombinant peptide is prepared comprising a first peptide and a second peptide. A spacer sequence of from about three to about thirty amino acids is optionally inserted between the first and second peptide. In a particular embodiment, the first peptide is HR-N10 and the second peptide is HR-C4, or vice versa.

### EXAMPLE 3

### Preparation of Variant Compounds

Other than natural peptides corresponding to fragments of the native S protein of SARS-CoV, variant compounds or analogs are also prepared. For variant peptides produced via recombinant technology, site-directed mutagenesis or random mutagenesis techniques are employed. Peptides are optionally produced by chemical synthetic methods.

A peptide of the invention is used as an inhibitor to block entry of a SARS coronavirus in a target cell. Such inhibition can be by prevention of fusion of a viral membrane and a target cell membrane. In a particular application, a peptide is used as a therapeutic agent that can reduce viral infectivity.

In an example, a first peptide is derived from or designed in relation to an S protein of SARS CoV or fragment thereof, for example an HR-N or HR-C domain peptide. For example, such first peptide is HR-N10 or HR-C4. A sequence of a first peptide can be modified so as to achieve a desirable functional attribute or to provide an alternative without a change in attribute. An attribute can include one or more of modified efficacy, specific activity, solubility, toxicity, or stability. The attribute of stability, for example, can relate to the ability to maintain a particular conformation, susceptibility to degradation such as by proteolysis, or to host effects such as clearance or parameters of distribution, metabolism, and excretion.

A peptide sequence or peptide product is modified in one or more of several ways. A peptide is modified so as to affect the alpha-helical propensity of the peptide product. A peptide sequence is modified by introduction of one or more cyclic bridges in a peptide sequence or peptide product. For example, a lactam bridge can be introduced into a peptide. A peptide is modified by modulation of the “a” and “d” position residues. A peptide is modified by chemical conjugation to polyethylene glycol (PEG) or pegylation of the sequence. A peptide is modified by removal and/or chemical alteration of sites sensitive to proteolysis. In a specific example, a proteolytically sensitive site is modified by acetylation.

Helical Propensity Changes.

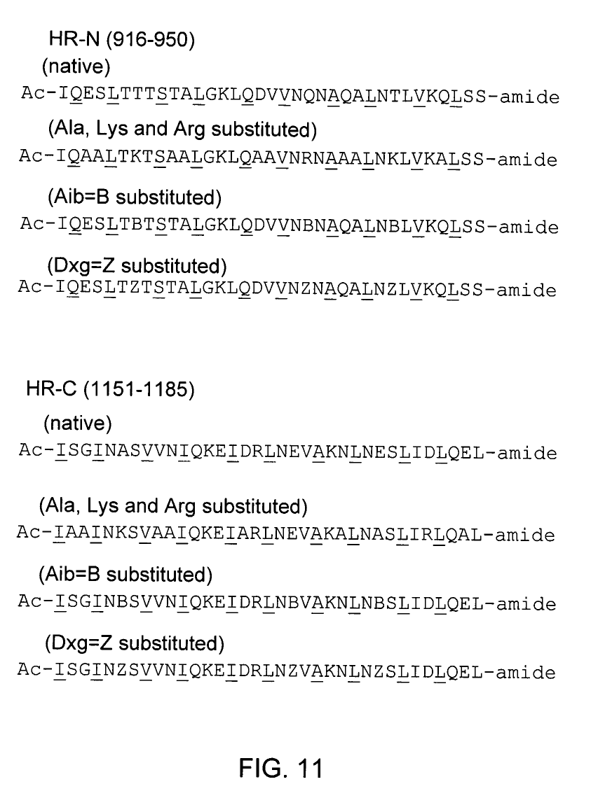

Regarding helical propensity changes, residues in the amino acid sequence of HR-N and HR-C can be substituted to increase the helical propensity (ability to form a helix) of the sequence and thus affect one or more attributes of the peptide such as stability, efficacy, or other attribute. One or more substitutions are made to residues occurring in the b, c, and f heptad positions with a molecule such as Ala, Lys, Arg, Aib (aminoisobutyric acid) or Dxg (dipropyl or dibutyl glycine). See FIG. 11 for examples of variously substituted peptides derived from native HR-N and HR-C sequences, including SEQ ID NO:51, SEQ ID NO:52, SEQ ID NO:53, SEQ ID NO:59, SEQ ID NO:60, and SEQ ID NO:61.

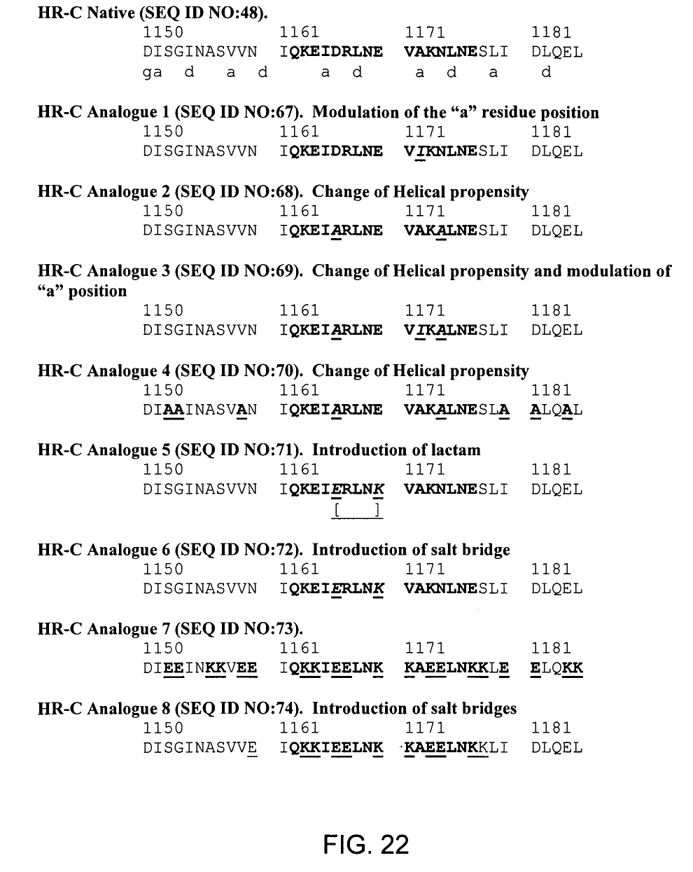

Other examples of HR-C analogs with ability for change of helical propensity include SEQ ID NO:68 and SEQ ID NO:70 (See FIG. 22).

Introduction of Lactam and other Cyclic Bridges.

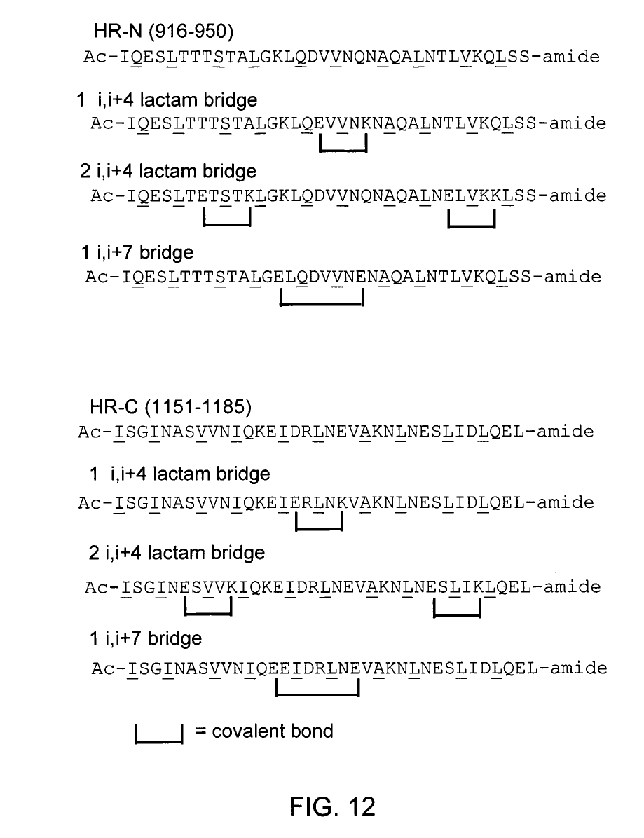

The formation of covalent bonds between side-chains of residues (lactam bridges) occurring on the outer helical face can significantly stabilize the helical conformation. For example, lactam bridge stabilization of alpha-helices in connection with the present invention is investigated. Parameters such as ring size, orientation, and positional effects are evaluated. Modified peptides are generated with one or more lactam bridges incorporated, for example into HR-N and HR-C sequences according to FIG. 12. Information such as the following is optionally utilized: a) (i, i+3) spacing gives a lactam between Lys and Glu with a 18-atom rings which is helix destabilizing regardless of position or location; b) (i,i+4) lactams with 21-atom rings are helix stabilizing but such stabilization can be dependent on orientation, that is, the Glu to Lys orientation is a desirable orientation; c) one Glu-Lys lactam bridge in the middle of the peptide is more effective at stabilizing helical structure than two Glu-Lys lactams positioned where one lactam bridge is at each end of the molecule; and d) lactam bridged alpha-helices dimerize better with lle (isoleucine) at position a and Leu at position d versus Val at position a and Leu at position d. Thus modulation of residues such as the hydrophobic core residues can be done independently or in connection with one or more bridges.

An example of an HR-C analog with a lactam bridge includes SEQ ID NO:71. An example of an HR-C analog with a salt bridge introduced includes SEQ ID NO:72. An example of an HR-C analog with multiple salt bridges includes SEQ ID NO:74. An example of an additional analog of an HR-C peptide is SEQ ID NO:73. FIG. 22 illustrates various HR-C analogs.

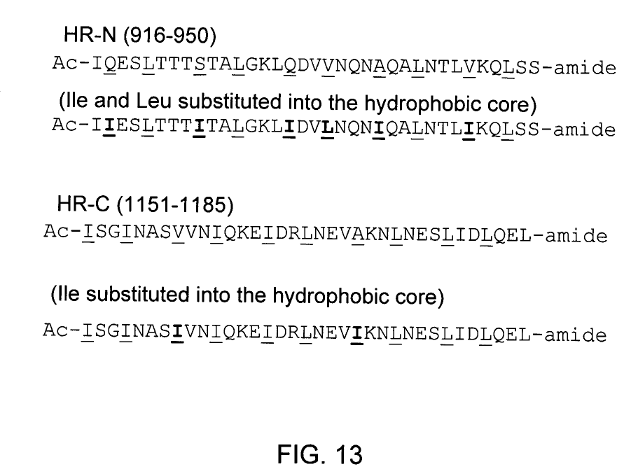

Modulation of the a and/or d residue positions. The residues occurring in the a and d heptad positions are significant for the folding and stability of the coiled-coil structure. By substituting the present a and d residues to those which are optimal for stability, for example, IIe (isoleucine) at position a and Leu (leucine) at position d, inhibitor analogs of HR-N and HR-C are produced which bind with higher affinity than the native sequences. See FIG. 13 for examples of substitution with either isoleucine (l/lle) or isoleucine and leucine (L/Leu).

Another example of an HR-C analog with modulation of the “a” and/or “d” residue positions includes SEQ ID NO:67 (see FIG. 22).

An example of an HR-C analog with modulation of the “a” and/or “d” residue positions and an ability for change of helical propensity includes SEQ ID NO:69 (see FIG. 22).

Generation of inhibitor peptides less than 35 amino acids. The chain length of a peptide compound is decreased from about 35 residues to a chain length of about 14 residues. Inhibitor compounds are also generated comprising from about 14 residues to about 35 residues. For example, a peptide is synthesized or expressed as a recombinant comprising any two heptad units, any three heptad units, or any four heptad units of the HR-N10 peptide, thereby generating 14 mers, 21 mers, and 28 mers. Therefore we generate inhibitor compounds, capable of demonstrating coiled-coil dimerization, with as few as about two heptads or a chain length of about 14 residues. See Table 3.

TABLE 3

Sub-peptides of HR-N10 and HR-C4 based on
heptad units.
Amino acids of S protein

| | | | | |
|---|---|---|---|---|
| Peptide name | Start | End | Length | Sequence |

| | | | | |
|---|---|---|---|---|
| HR-N10 | 916 | 950 | 35 | IQESLTTTSTALGKLQDV |
| | | | | VNQNAQALNTLVKQLSS |
| Heptad 1 | | | | IQESLTT |
| Heptad 2 | | | | TSTALGK |
| Heptad 3 | | | | LQDVVNQ |
| Heptad 4 | | | | NAQALNT |
| Heptad 5 | | | | LVKQLSS |

2 heptad units

| | | | | |
|---|---|---|---|---|
| 1,2 | 1151 | 1164 | 14 | IQESLTTTSTALGK |
| 2,3 | 923 | 936 | 14 | TSTALGKLQDVVNQ |
| 3,4 | 930 | 943 | 14 | LQDVVNQNAQALNT |
| 4,5 | 937 | 950 | 14 | NAQALNTLVKQLSS |

3 heptad units

| | | | | |
|---|---|---|---|---|
| 1,2,3 | 1151 | 1171 | 21 | IQESLTTTSTALGKLQDV |
| | | | | VNQ |
| 2,3,4 | 923 | 943 | 21 | TSTALGKLQDVVNQNAQA |
| | | | | LNT |
| 3,4,5 | 930 | 950 | 21 | LQDVVNQNAQALNTLVKQ |
| | | | | LSS |

4 heptad units

| | | | | |
|---|---|---|---|---|
| 1,2,3,4 | 1151 | 1178 | 28 | IQESLTTTSTALGKLQDV |
| | | | | VNQNAQALNT |
| 2,3,4,5 | 923 | 950 | 28 | TSTALGKLQDVVNQNAQA |
| | | | | LNTLVKQLSS |
| HR-C4 | 1151 | 1185 | 35 | ISGINASVVNIQKEIDRL |
| | | | | NEVAKNLNESLIDLQEL |
| Heptad 1 | | | | ISGINAS |
| Heptad 2 | | | | VVNIQKE |
| Heptad 3 | | | | IDRLNEV |
| Heptad 4 | | | | AKNLNES |
| Heptad 5 | | | | LIDLQEL |

2 heptad units

| | | | | |
|---|---|---|---|---|
| 1,2 | 1151 | 1164 | 14 | ISGINASVVNIQKE |
| 2,3 | 1158 | 1171 | 14 | VVNIQKEIDRLNEV |
| 3,4 | 1165 | 1178 | 14 | IDRLNEVAKNLNES |
| 4,5 | 1172 | 1185 | 14 | AKNLNESLIDLQEL |

3 heptad units

| | | | | |
|---|---|---|---|---|
| 1,2,3 | 1151 | 1171 | 21 | ISGINASVVNIQKEIDRL |
| | | | | NEV |
| 2,3,4 | 1158 | 1178 | 21 | VVNIQKEIDRLNEVAKNL |
| | | | | NES |
| 3,4,5 | 1165 | 1185 | 21 | IDRLNEVAKNLNESLIDL |
| | | | | QEL |

4 heptad units

| | | | | |
|---|---|---|---|---|
| 1,2,3,4 | 1151 | 1178 | 28 | ISGINASVVNIQKEIDRL |
| | | | | NEVAKNLNES |
| 2,3,4,5 | 1158 | 1185 | 28 | VVNIQKEIDRLNEVAKNL |
| | | | | NESLIDLQEL |

Pegylation. Molecules are modified by the addition of the polymer polyethylene glycol (PEG). PEG addition or conjugation is performed as known in the art. Peptides with PEG can have one or more properties changed. For example, a PEG-peptide can be less prone to degradation such as by proteolysis. The weight. This difference may indicate that the HR-N region naturally associates as a trimer, but in the absence of the HR-C region (or other parts of the native sequence) the hydrophobic residues occurring in the e and g positions are exposed. To sequester these residues from the aqueous environments, the protein can switch from a trimer to a tetramer state. In agreement with this proposal is the observation that more interfacial surface area, in particular the e and g positions are buried in a tetrameric coiled-coil structure relative to a trimeric or dimeric coiled-coil (64).

The HR-C region in isolation showed a very concentration-dependent alpha-helical coiled-coil structure which at very high concentration (>500 μM) indicated only about 24 helical residues. The truncation studies showed that the N-terminal four residues could be deleted without adversely affecting the structure. Additionally, both sedimentation equilibrium and gel electrophoresis showed that the entire HR-C region alone associates as a homotrimer. Consistent with the helicity data, stability analysis showed that HR-C1 was of low stability with 50% of its structure unfolded at 33° C. This and the above characteristic is surprising, considering that 9 out of the 10 heptad a and d core positions contain large optimal hydrophobic residues (e.g. I, I, V, I, I, L, L, L, L), and no apparent intermolecular electrostatic repulsions can be predicted. The low stability, however, does fit with a general mechanism of type-1 viral fusion proteins, where the C-terminal HR separates in order to form a complex with the N-terminal HR. In agreement with this is the observation that there are no possible inter-chain electrostatic interactions that can occur between HR-C peptide chains. Such electrostatic interactions can add considerable stability to coiled-coils and stabilize their ability to stay associated. See (65) and references therein.

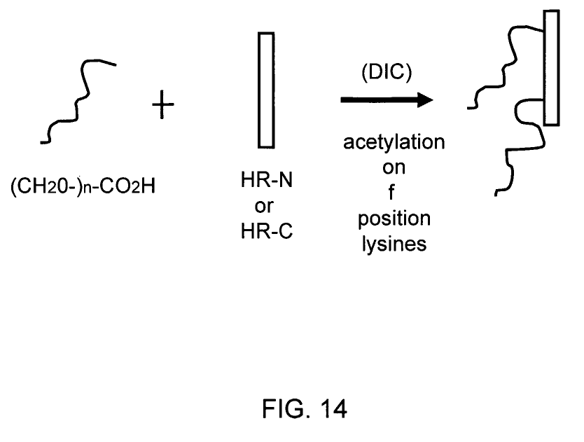

Upon mixing the HR-C1 peptide with several truncated HR-N region peptides, HR-C1 associated preferentially with HR-N10 to form a stable α-helical 6-stranded complex, comprising of a 3:3 mole ratio of HR-N10:HR-C1. Additionally, the selective disulfide bridge experiments indicated that the relative orientation of HR-N10 and HR-C1 peptides in the complex were anti-parallel relative to one another. The peptide mapping study revealed that a 35-residue length is desirable and may be optimal for the binding interaction with HR-C1, as shifting either 7 residues toward the N or C-terminus of HR-N (HR-9 or HR-11) caused significant effective lifetime, for example the half-life, of a PEG-peptide can be enhanced. The immunogenicity of a PEG-peptide can be decreased. In a specific example, modified HR-N and HR-C inhibitor peptides are generated wherein PEG is acetylated onto lysine residues substituted into the b, c, or f heptad positions along the sequence. See FIG. 14.

Acetylation/mutation of sensitive sites. Residues which are prone to proteolytic cleavage, for example lysine in connection with trypsin, are optionally modified by acetylation using acetic anhydride or its equivalent. Such residues can also be mutated.

Use of Natural Amino Acids and other Building Blocks.

Peptides can be prepared using natural amino acids as building blocks for either recombinant expression or synthetic production. It is well known in the biological arts that certain amino acid substitutions can be made in protein sequences without affecting the function of the protein. Generally, conservative amino acids are tolerated without affecting protein function. Similar amino acids can be those that are similar in size and/or charge properties, for example, aspartate and glutamate and isoleucine and valine are both pairs of similar amino acids. Here, however, substituted peptides are further evaluated and selected regarding the affect of a substitution on the ability to maintain an alpha-helical coil structure or so as to facilitate a capability of forming a trimeric conformation, including a homotrimer, or a six helix bundle conformation, including a heterologous bundle.

Peptides are also prepared using at least one chemical moiety such as a non-proteinogenic amino acid, unnatural amino acid, peptidomimetic unit, peptoid, beta amino acid, or derivatized amino acid molecule. The moiety is selected so as to maintain an alpha-helical coil structure or so as to facilitate a capability of forming a trimeric conformation, including a homotrimer, or a six helix bundle conformation, including a heterologous bundle. For example, the moiety and/or substitution is selected and integrated in an HR-N10 or HR-C4 peptide derivative so as to maintain an alpha-helical structure. Alternatively, the moiety and/or substitution is selected and a peptide is prepared so as to maintain a functional ability to inhibit infectivity or cell-cell fusion in connection with a SARS coronavirus or a SARS coronavirus S protein.

Any of the peptides described herein may, additionally, have a non-peptide macromolecular carrier group covalently attached to their amino and/or carboxy termini. Such macromolecular carrier groups may include, for example, lipid-fatty acid conjugates, polyethylene glycol, or carbohydrates.

### EXAMPLE 4

### Screening and Analysis of Peptide Inhibitors

Biophysical Properties.

The peptides or modified peptides are evaluated or screened regarding one or more biophysical properties. Modified peptides are evaluated with respect to stability. In a particular example, compounds are selected that demonstrate improved stability relative to that of peptides with native sequences. Stability can be defined here by the temperature midpoint unfolding transitions or Gdn-HCl denaturation unfolding transitions. See, e.g., Pace, C. N. 1986, Determination and analysis of urea and guanidine hydrochloride denaturation curves, Methods Enzymol. 131:266-280; Weber, P. C. and F. R. Salemme, 2003 Feb, Applications of calorimetric methods to drug discovery and the study of protein interactions, Curr. Opin. Struct. Biol. 13: 115-121.

Bioassays. Bioassays are performed to assess attributes of compounds such as peptides, for example relating to the ability to modify fusion and infectivity.

Cell-cell fusion assay. The inhibitory ability of the peptides (and analogs) is determined using a cell-to-cell fusion assay. Cell-to-cell fusion assays are performed by transfecting a receptor negative cell line with recombinantly expressed anchored S protein. When overlaid with cells expressing receptor, at least partial cell fusion occurs. Cell fusion is detected with a calorimetric assay. A first cell line expresses T7 polymerase, and a second cell line expresses beta-galactosidase under control of the T7 promoter; fusion results in blue cells. Peptides are added in increasing concentrations to determine the inhibition of the membrane fusion step. For another cell fusion assay system, see that of Bosch (19) which is adapted as necessary.

Cell infectivity assay. Cell infectivity assays are performed. Vero cells are inoculated with SARS-COV at a multiplicity of infection of 0.2 PFU/cell in the absence of inhibitor. After 2 hours when the virus is into the cells, a putative inhibitor compound is added. Cells are fixed at 12 hours and 24 hours, and immunofluorescence is performed to detect cells that have synthesized viral antigens. The results are evaluated to identify inhibitor compounds by determining if the S protein on the cell membrane is inhibited, for example in the ability to achieve cell to cell spread of infection or cell fusion. The results are also evaluated regarding whether newly made virus is prevented from infecting other cells which depends on fusion of the viral envelope with the plasma membrane of an uninfected cell. In addition, Vero cells are inoculated with SARS-CoV at a multiplicity of infection of 10 PFU/cell in the absence of inhibitor. Under these conditions (with no inhibition) infected cells are antigen positive by 6 hours post-infection. The percent of infected cells in the presence of an inhibitor relative to control (absence of an inhibitor) is determined to assess inhibition of initial virus infection.

For peptide compounds where inhibition is detected, a dose-response inhibitory activity is determined. Peptides having substantial inhibitory activity are used to generate additional analogs with further modification. These analogs are optimized for one or more properties such as polypeptide chain length, hydrophobicity, helical propensity and electrostatic interactions.

Peptides and analogs are optionally generated by providing one or more constraints such as a covalent constraint. A covalent constraint can enhance the stability of a conformation such as the helical conformation. Constrained peptides can exhibit a desirable conformational entropy profile, for example an entropy that is lower than that for an unconstrained counterparts or peptide. A constrained peptide can adopt a single specific bioactive conformation and show a smaller decrease in entropy on binding. A constrained peptide can demonstrate an enhancement biological activity such as receptor binding avidity and affinity. Such enhancement can occur by one or more of (1) stabilizing the biologically active conformer; (2) decreasing degradation; and (3) improving biological selectivity through reduction or elimination of bioactive conformers in connection with undesired biological responses.

### EXAMPLE 5

### Peptides as Immunogenic Agents

Peptides herein are used to stimulate an immune response in a host organism. For example, a peptide is used to act as a vaccine in a human to aid in preventing or blocking fusion, entry, infection, or disease in connection with SARS coronavirus. An immune response comprising an antibody response and a cellular response is thereby generated upon introduction of an effective amount of an immunogenic form of a peptide. For example, antibodies raised against SARS-CoV HR-N and HR-C peptide antigens are useful in at least partially blocking SARS-COV entry by binding to the corresponding conserved sequences in the SARS-COV S protein, thereby neutralizing free virus and preventing fusion of receptor bound virus.

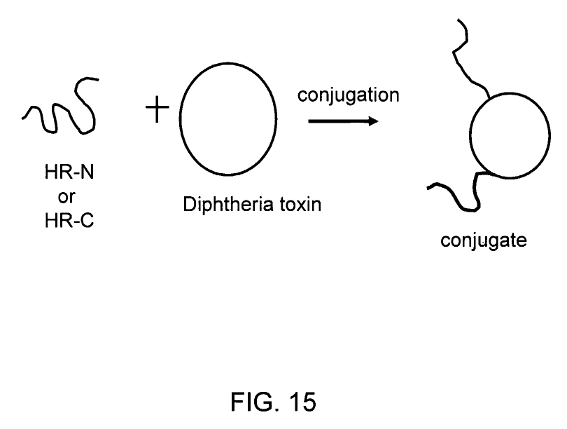

In an embodiment, a peptide is conjugated to a carrier protein. A carrier protein is selected from those known in the art. Diphtheria toxin is optionally the carrier protein. The conjugated peptide is used directly to immunize a subject. An adjuvant is optionally used with the conjugated peptide as known in the art for potentiation of an immune response. For example, an adjuvant is selected so as to enhance an IgG response. In another approach, the peptides are linked to a carrier protein, for example through an amino or carboxyl group of the peptide sequence or via a cysteine residue added to a peptide sequence terminus. See FIG. 15.

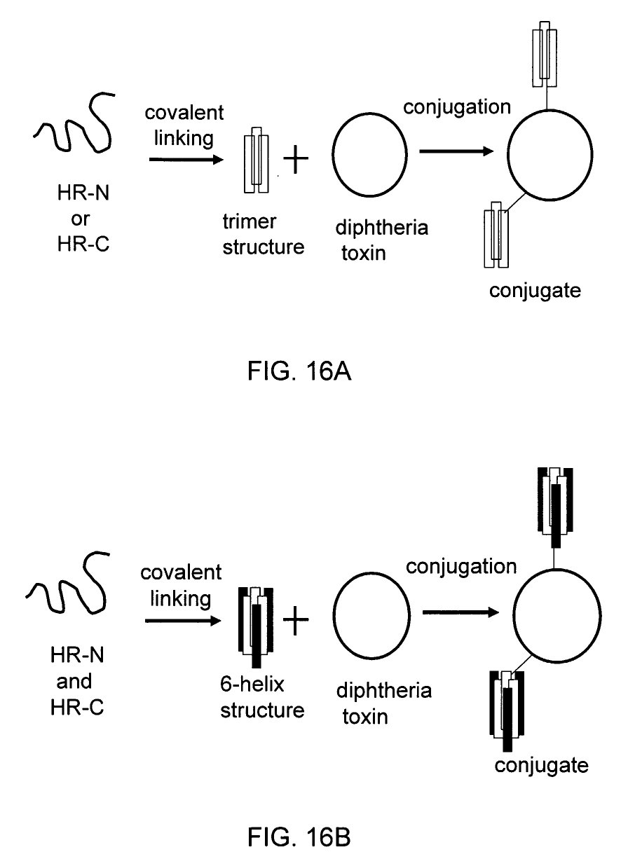

Immunogens comprising a trimer structure. Each of the peptide regions, for example HR-N or HR-C, is cross-linked together in a trimeric oligomeric state. This trimeric structure is used as a native-like coiled-coil antigen. For example, an HR-10 peptide is used to generate a homotrimer. The cross-linked molecules are optionally conjugated to a carrier protein. Such a conjugated composition is further optionally formulated or administered with an adjuvant. It is believed that the stabilized trimer antigen can induce an immune response such as an antibody response that is directed towards the relevant fusion competent conformation of a SARS coronavirus S protein. See FIG. 16.

Immunogens comprising a six-helix bundle structure. A covalently linked six-stranded molecule of three HR-N and three HR-C peptides is produced. This six-stranded structure is optionally conjugated to a carrier protein for immunization. See FIG. 16.

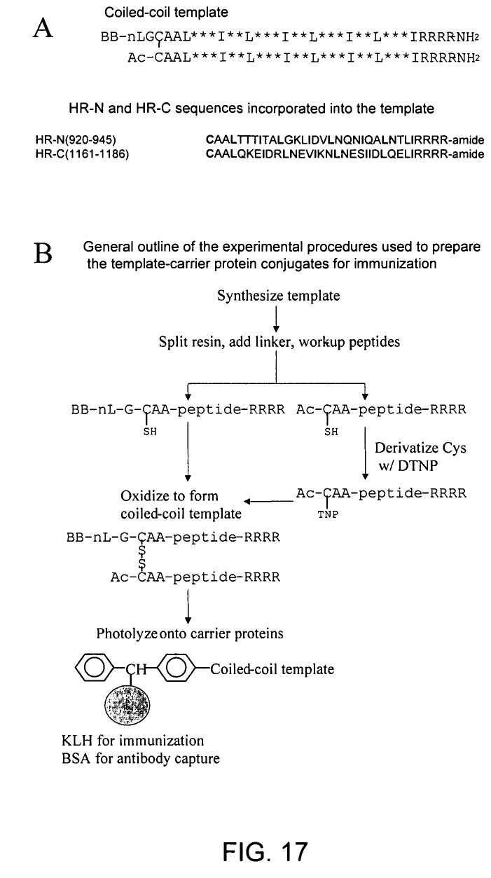

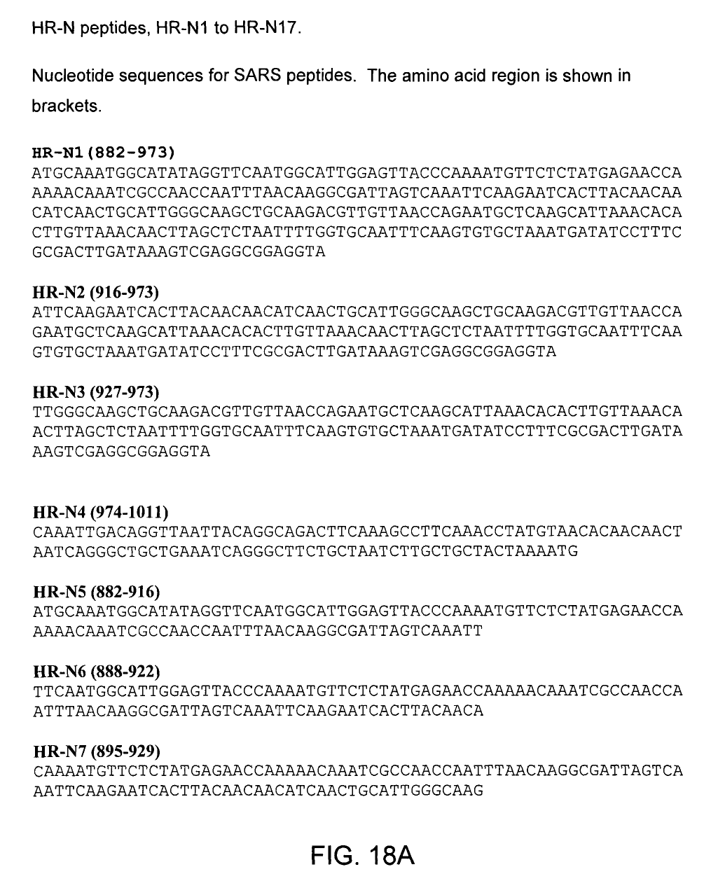

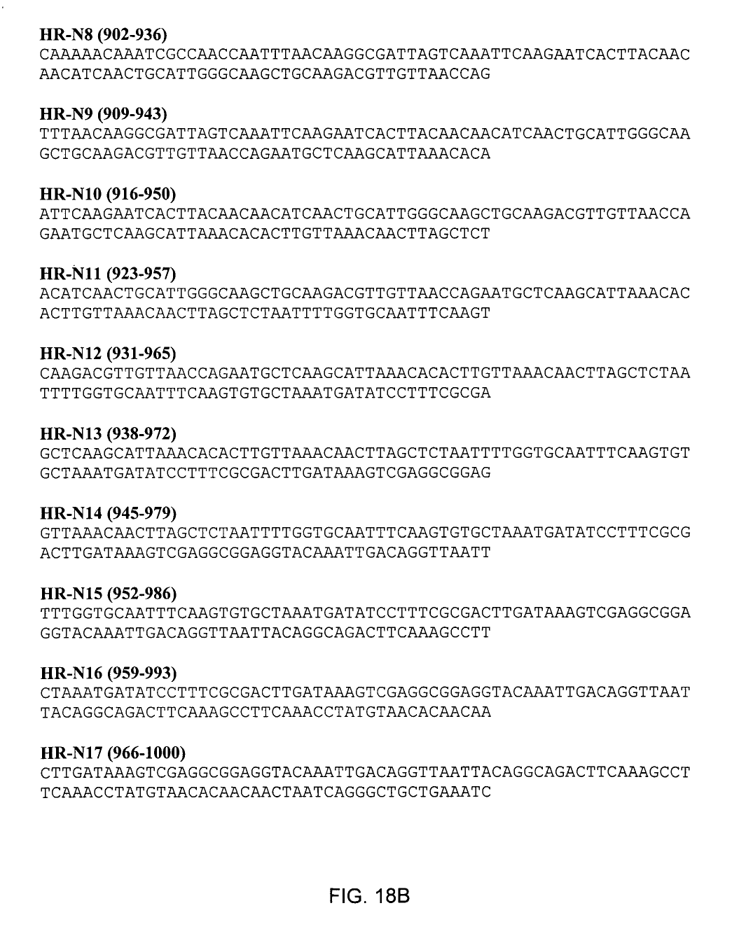

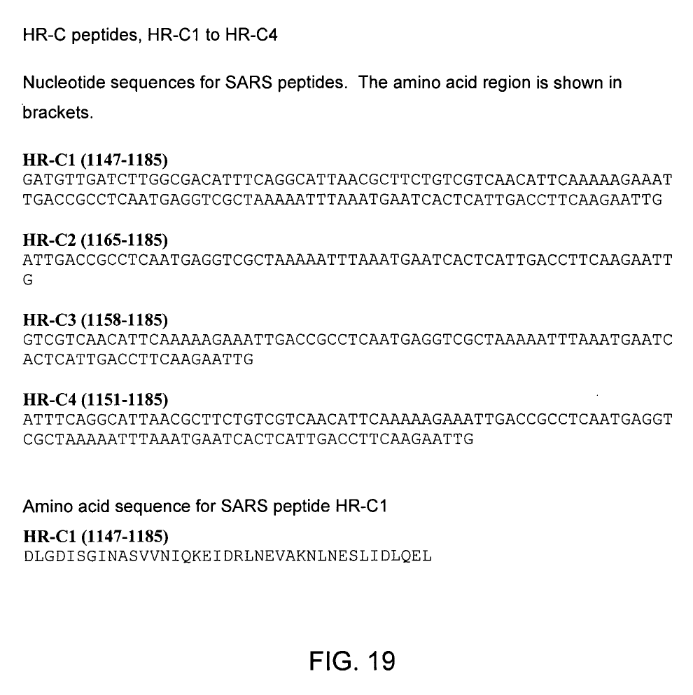

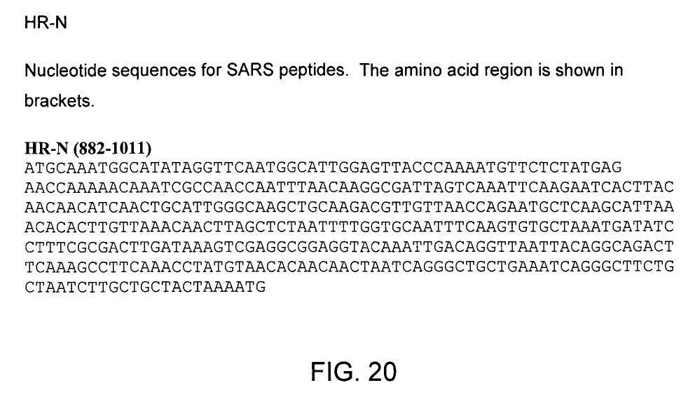

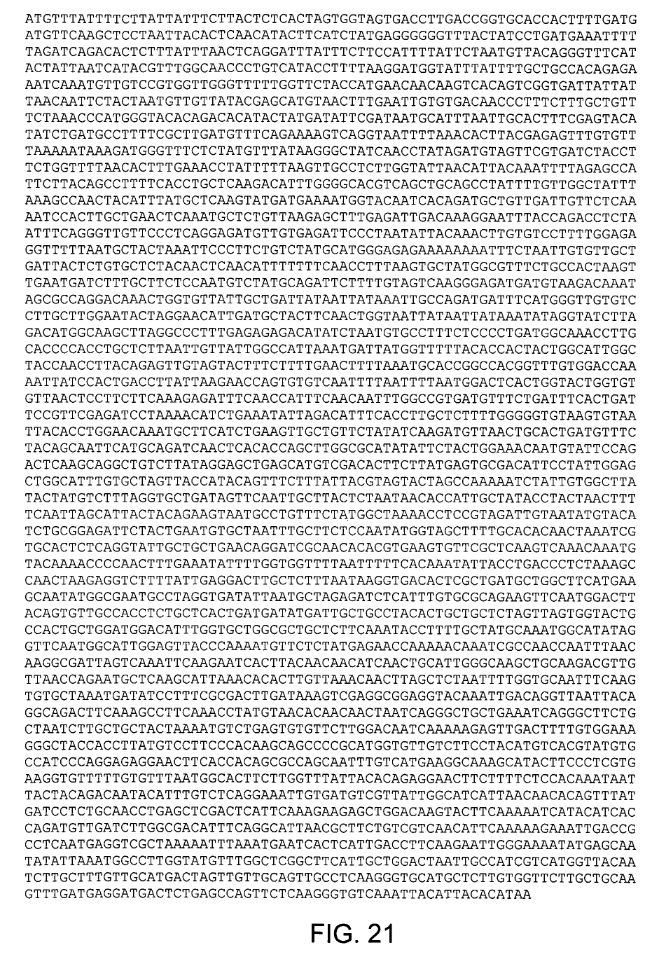

Immunogens comprising a helix template. A method is developed for the design of stabilized α-helical immunogens as a means of generating antibodies specific for helical protein segments. The method employs a two-stranded α-helical parallel coiled-coil as a template. The template and its preparation are shown in FIG. 17. Sequences of HR-N and HR-C are incorporated into the template, thereby producing compositions suitable for induction or stimulation of an immune response such as an antibody response. Using the helix template technology, specific antibodies are generated that target helical regions of proteins. Such antibodies can react against the native sequences of the SARS S protein, particularly against certain conformations of the native S protein relevant to fusion or entry such as the trimeric or six-stranded structures.

Tandem repeat antigens. For peptides developed as immunogens, each of the sequences can be repeated. For example, a tandem peptide repeat can have the form of (peptide)ₙ, wherein the peptide is present a total of n times. The repeat total is selected as is known in the art. In a particular example, n is from about two to about eight. In another example, an upper bound for n is determined empirically and is estimated as known in the art. It is believed that a tandem repeat approach can further increase the immunogenicity or antigenicity of a peptide.

Any peptide or other structure comprising a peptide (for example, a trimeric structure, six-stranded structure, or other structure) is optionally conjugated to a carrier protein. Such a conjugated composition is further optionally formulated with or administered in connection with an adjuvant.

The peptides may be formulated with a suitable adjuvant in order to enhance the immunological response. Such adjuvants may include, but are not limited to mineral gels such as aluminum hydroxide; surface active substances such as lysolecithin, pluronic polyols, polyanions; other peptides; oil emulsions; and potentially useful human adjuvants such as BCG and Corynebacterium parvum. Many methods may be used to introduce the vaccine formulations described here. These methods include but are not limited to oral, intradermal, intramuscular, intraperitoneal, intravenous, subcutaneous, mucosal, respiratory, and intranasal routes.

A polyclonal or monoclonal antibody molecule or population generated in connection with a peptide of the present invention is evaluated regarding one or more properties such as specificity, affinity, avidity, kinetic parameter, and biological activity. Techniques are used as known in the art, for example Western blotting, ELISA, and analysis using surface plasmon resonance technology (such as with a BiaCore device). For example, the biological activity of an antibody is tested for the ability to neutralize or inhibit infectivity or fusion by a SARS coronavirus or a SARS coronavirus protein in the context of a cell-cell fusion assay or viral infectivity assay. Antibodies are optionally raised initially in mice and rabbits for analysis.

A nucleotide molecule encoding a peptide of the invention can also be used for a genetic immunization approach as is known in the art.

### EXAMPLE 6

### Antibody Therapeutic Agent

The SARS-CoV HR-N and HR-C peptides are used to generate humanized monoclonal and polyclonal antibodies for passive immunotherapy treatment of SARS. An effective amount of an antibody preparation is provided and administered to a human subject suspected of a SARS coronavirus infection. The antibody can operate to reduce the ability of a SARS coronavirus S protein to make a conformation that is competent for fusion or entry in a target cell. Therefore an effective concentration of polyclonal or monoclonal antibodies raised against the peptides of the invention is administered to a host so that uninfected cells are less prone to becoming infected by SARS. The exact concentration of such antibodies will vary according to each specific antibody preparation, but may be determined using standard techniques well known to those of ordinary skill in the art. Administration of the antibodies may be accomplished using a variety of techniques, including, but not limited to those described herein.

### EXAMPLE 7

### Diagnostic Application of Antibodies

Antibodies raised against the SARS HR-N and HR-C peptides are used in diagnosis to test for the presence of free virus in tissue or fluid samples.

### EXAMPLE 8

### Antibodies as Screening Reagent

Antibodies are used in a drug screening procedure to identify compounds that inhibit the formation of the 6-helix bundle fusion active core or the function of fusion or entry. The conformation-specific antibodies are used to detect the presence of the HR-N/HR-C 6-helix bundle structure. The binding of a test compound such as a drug to this structure, for example so as to disrupt this structure, is associated with the loss of an antibody reagent. Such loss can be by competitive inhibition due to displacement of the antibody by the test compound. Therefore the identification of a test compound capable of relevant binding is conducted using immunological procedures as known in the art.

### EXAMPLE 9

### Derivatives of HR peptides HR-N10 and HR C4

An HR peptide such as HR-N10 is extended in either direction of the N/amino terminus or C/carboxy terminus. A peptide derived from HR-N10 is prepared by synthesizing a peptide beginning with any of HR-N amino acids 909 to 915 and ending with any of HR-N amino acids 951 to 957. For example, a derivative of HR-N10 is prepared by addition of about one heptad unit at one terminus or both termini. Other derivatives are prepared using less than a full heptad unit, for example from one to six amino acids.

An HR peptide such as HR-C4 is deleted from its N terminus so that from one to six amino acids are deleted. A peptide derived from HR-C4 is prepared by synthesizing a peptide beginning with any of HR-C amino acids 1152 to 1157. Such a peptide can also be perceived as being derived from HR-C3 with an earlier beginning point of the amino acid sequence.

The peptide derivative is tested for stability. The peptide is tested for ability to form a trimeric conformation or a six helix bundle conformation in a complex with an HR-N or HR-C peptide or native protein. The peptide is tested for ability to inhibit cell-cell fusion or infectivity.

### EXAMPLE 10

### Therapeutic Applications of Peptides

A further object of the invention is to provide therapeutic compositions, suitable for human or veterinary pharmaceutical use, comprising a peptide, peptides, or complexes of a peptide or peptides of the present invention and a suitable pharmacological carrier. Such therapeutic compositions can be formulated as understood in the art for administration. For example, administration may be applied via oral, intradermal, intramuscular, intraperitoneal, intravenous, subcutaneous, topical, mucosal, respiratory, and intranasal routes. For a respiratory route, an aerosol can be delivered from a nebulization formula, or an atomization spray formulation can be prepared. A therapeutic composition can control and/or prevent infection by a coronavirus, particularly a SARS coronavirus.

Effective dosages of the peptides of the invention to be administered may be determined through procedures well known to those in the art which address such parameters as biological half-life, bioavailability, and toxicity. For example, peptide HR-N10 may prove efficacious in vivo at doses required to achieve circulating levels of about 10 ng per ml of peptide.

Pharmaceutical compositions are formulated to contain a therapeutically effective amount of a composition of the invention and a pharmaceutically acceptable carrier appropriate for the route of administration. A composition of the invention can include, for example, a peptide of the invention or salt thereof.

A therapeutic form can comprise a carrier hydrogel composition of a polymer material and an effective dose of a peptide. The polymer materials used in the carrier hydrogel composition can preferably have reverse gelation properties and exist as a liquid, aqueous solution at temperatures below physiological temperatures (e.g., below the body temperature of a patient) but form hydrogels under physiological conditions (e.g., at temperatures at or near the body temperature of a patient). The carrier hydrogel compositions may thus be administered to a patient by injection while they are in a liquid state. Upon administration the carrier hydrogel compositions then form hydrogels with a peptide embedded therein. The peptide or peptides are thereby released with improved pharmacokinetic properties and bioavailability.

### EXAMPLE 11

### Applicability to SARS Coronaviruses

Compositions and methods of the invention are applicable to SARS coronaviruses generally. For example, the nucleotide sequences of NCBI Accession Numbers in Table 4 and protein sequences of NCBI Accession Numbers in Table 5 are used in embodiments of the invention.

TABLE 4
SARS coronavirus nucleotide sequences.

| Accession No. | Description |
|---|---|
| 1: AY525636 | SARS coronavirus GD03T0013 spike glycoprotein gene, complete cds |
| 2: NC_004718 | SARS coronavirus, complete genome |
| 3: AY443094 | SARS coronavirus CUHK-L2 spike glycoprotein and orf3 genes, partial cds |
| 4: AY443093 | SARS coronavirus CUHK-L2 spike glycoprotein gene, partial cds |
| 5: AH013657 | SARS coronavirus CUHK-L2 isolate CUHK-L2, a Hong Kong isolate that |
| | antedated the Metropole Hotel case cluster orf1ab polyproteins, spike |
| | glycoproteins, orf3, orf4, and orf3 genes, partial cds; small envelope E |
| | protein, membrane glycoprotein M, orf7, orf8, orf9, orf10, and orf11 genes, |
| | complete cds; nucleocapsid protein gene, partial cds; and orf13 gene, |
| | complete cds |
| 6: AY390556 | SARS coronavirus GZ02, complete genome |
| 7: AY508724 | SARS coronavirus NS-1, complete genome |
| 8: AY502932 | SARS coronavirus TW9, complete genome |
| 9: AY502931 | SARS coronavirus TW8, complete genome |
| 10: AY502930 | SARS coronavirus TW7, complete genome |
| 11: AY502929 | SARS coronavirus TW6, complete genome |
| 12: AY502928 | SARS coronavirus TW5, complete genome |
| 13: AY502927 | SARS coronavirus TW4, complete genome |
| 14: AY502926 | SARS coronavirus TW3, complete genome |
| 15: AY502925 | SARS coronavirus TW2, complete genome |
| 16: AY502924 | SARS coronavirus TW11, complete genome |
| 17: AY502923 | SARS coronavirus TW10, complete genome |
| 18: AY463059 | SARS coronavirus ShanghaiQXC1, complete genome |
| 19: AY463060 | SARS coronavirus ShanghaiQXC2, complete genome |
| 20: AY310120 | SARS coronavirus FRA, complete genome |
| AY485278 | SARS coronavirus Sino3-11, complete genome |
| 22: AY485277 | SARS coronavirus Sino1-11, complete genome |
| 23: AY345988 | SARS coronavirus CUHK-AG03, complete genome |
| 24: AY345987 | SARS coronavirus CUHK-AG02, complete genome |
| 25: AY345986 | SARS coronavirus CUHK-AG01, complete genome |
| 26: AY282752 | SARS coronavirus CUHK-Su10, complete genome |
| 27: AY429079 | SARS coronavirus BJ302 clone 8 spike glycoprotein gene, complete cds |
| 28: AY429078 | SARS coronavirus BJ302 clone 7 spike glycoprotein gene, complete cds |
| 29: AY429077 | SARS coronavirus BJ302 clone 6 spike glycoprotein gene, complete cds |
| 30: AY429076 | SARS coronavirus BJ302 clone 5 spike glycoprotein gene, complete cds |
| 31: AY429075 | SARS coronavirus BJ302 clone 4 spike glycoprotein gene, complete cds |
| 32: AY429074 | SARS coronavirus BJ302 clone 3 spike glycoprotein gene, complete cds |
| 33: AY429073 | SARS coronavirus BJ302 clone 2 spike glycoprotein gene, complete cds |
| 34: AY429072 | SARS coronavirus BJ302 clone 1 spike glycoprotein gene, complete cds |
| 35: AY427439 | SARS coronavirus AS, complete genome |
| 36: AY323977 | SARS coronavirus HSR 1, complete genome |
| 37: AY276489 | SARS coronavirus GD01, complete genome |
| 38: AY278741 | SARS coronavirus Urbani, complete genome |
| 39: AP006561 | SARS coronavirus TWY genomic RNA, complete genome |
| 40: AP008580 | SARS coronavirus TWS genomic RNA, complete genome |
| 41: AP006559 | SARS coronavirus TWK genomic RNA, complete genome |
| 42: AP006558 | SARS coronavirus TWJ genomic RNA, complete genome |
| 43: AP006557 | SARS coronavirus TWH genomic RNA, complete genome |
| 44: AY278554 | SARS coronavirus CUHK-W1, complete genome |
| 45: AY322207 | SARS coronavirus Shanhgai LY orf1ab polyprotein gene, partial cds; spike |
| | glycoprotein gene, complete cds; and Orf3a gene, partial cds |
| 46: AH012999 | SARS coronavirus Shanhgai LY orf1a polyprotein, orf1ab polyprotein, orf1a |
| | polyprotein, orf1ab polyprotein, and orf1ab polyprotein genes, partial cds; |
| | spike glycoprotein gene, complete cds; Orf3a and Orf7a genes, partial cds; |
| | and Orf7b, Orf8A, Orf8b, and nucleocapsid protein genes, complete cds |
| 47: AY323976 | SARS coronavirus ZJ01 spike glycoprotein mRNA, complete cds |
| 48: AY291315 | SARS coronavirus Frankfurt 1, complete genome |
| 49: AY274119 | SARS coronavirus TOR2, complete genome |
| 50: AY291451 | SARS coronavirus TW1, complete genome |
| 51: AY278488 | SARS coronavirus BJ01, complete genome |

TABLE 5
SARS coronavirus protein sequences by NOBI Accession Number.

| Accession No. | Description |
|---|---|
| 1: AAS10463 | spike glycoprotein [SARS coronavirus GD03T0013] |
| 2: NP_828851 | E2 glycoprotein precursor; putative spike glycoprotein [SARS |
| | coronavirus] |
| 3: P59594 | E2 glycoprotein precursor (Spike glycoprotein) (Peplomer protein) |
| 4: AAS01063 | spike glycoprotein [SARS coronavirus CUHK-L2] |
| 5: AA501062 | spike glycoprotein [SARS coronavirus CUHK-L2] |
| 6: AAS00003 | spike glycoprotein [SARS coronavirus GZ02] |
| 7: AAR91586 | spike glycoprotein S [SARS coronavirus NS-1] |
| 8: AAR87600 | putative spike glycoprotein [SARS coronavirus TW9] |
| 9: AAR87589 | putative spike glycoprotein [SARS coronavirus TW8] |
| 10: AAR87578 | putative spike glycoprotein [SARS coronavirus TW7] |
| 11: AAR87567 | putative spike glycoprotein [SARS coronavirus TW6] |
| 12: AAR87556 | putative spike glycoprotein [SARS coronavirus TW5] |
| 13: AAR87545 | putative spike glycoprotein [SARS coronavirus TW4] |
| 14: AAR87534 | putative spike glycoprotein [SARS coronavirus TW3] |
| 15: AAR87523 | putative spike glycoprotein [SARS coronavirus TW2] |
| 16: AAR87512 | putative spike glycoprotein [SARS coronavirus TW11] |
| 17: AAR87501 | putative spike glycoprotein [SARS coronavirus TW10] |
| 18: AAR86788 | spike [SARS coronavirus ShanghaiQXC1] |
| 19: AAR86775 | spike [SARS coronavirus ShanghaiQXC2] |
| 20: AAP50485 | spike glycoprotein [SARS coronavirus FRA] |
| 21: AAR23258 | spike protein [SARS coronavirus Sino3-11] |
| 22: AAR23250 | spike protein [SARS coronavirus Sino1-11] |
| 23: AAP94759 | putative E2 glycoprotein precursor [SARS coronavirus CUHK-AG03] |
| 24: AAP94748 | putative E2 glycoprotein precursor [SARS coronavirus CUHK-AG02] |
| 25: AAP94737 | putative E2 glycoprotein precursor [SARS coronavirus CUHK-AG01] |
| 26: AAP30713 | putative spike glycoprotein [SARS coronavirus CUHK-Su10] |
| 27: AAR07631 | spike glycoprotein [SARS coronavirus BJ302] |
| 28: AAR07630 | spike glycoprotein [SARS coronavirus BJ302] |
| 29: AAR07629 | spike glycoprotein [SARS coronavirus BJ302] |
| 30: AAR07628 | spike glycoprotein [SARS coronavirus BJ302] |
| 31: AAR07627 | spike glycoprotein [SARS coronavirus BJ302] |
| 32: AAR07626 | spike glycoprotein [SARS coronavirus BJ302] |
| 33: AAR07625 | spike glycoprotein [SARS coronavirus BJ302] |
| 34: AAR07624 | spike glycoprotein [SARS coronavirus BJ302] |
| 35: AAQ94060 | spike protein S [SARS coronavirus AS] |
| 36: AAP72986 | spike protein S [SARS coronavirus HSR 1] |
| 37: AAP51227 | spike glycoprotein S [SARS coronavirus GD1] |
| 38: AAP13441 | S protein [SARS coronavirus Urbani] |
| 39: BAC81404 | spike protein S [SARS coronavirus TWY] |
| 40: BAC81390 | spike protein S [SARS coronavirus TWS] |
| 41: BAC81376 | spike protein S [SARS coronavirus TWK] |
| 42: BAC81362 | spike protein S [SARS coronavirus TWJ] |
| 43: BAC81348 | spike protein S [SARS coronavirus TWH] |
| 44: AAP13567 | putative E2 glycoprotein precursor [SARS coronavirus CUHK-W1] |
| 45: AAP82968 | spike glycoprotein [SARS coronavirus Shanhgai LY] |
| 46: AAP73417 | spike glycoprotein [SARS coronavirus ZJ01] |
| 47: AAP33697 | spike protein S [SARS coronavirus Frankfurt 1] |
| 48: AAP41037 | spike glycoprotein [SARS coronavirus Tor2] |
| 49: AAP37017 | putative spike glycoprotein S [SARS coronavirus TW1] |
| 50: AAP30030 | spike glycoprotein S [SARS coronavirus BJ01] |

### REFERENCES IN CONNECTION WITH EXAMPLE 1

1. Drosten, C., Gunther, S., Preiser, W., van der Werf, S., Brodt, H. R., Becker, S., Rabenau, H., Panning, M., Kolesnikova, L., Fouchier, R. A., Berger, A., Burguiere, A. M., Cinatl, J., Eickmann, M., Escriou, N., Grywna, K., Kramme, S., Manuguerra, J. C., Muller, S., Rickerts, V., Sturmer, M., Vieth, S., Klenk, H. D., Osterhaus, A. D., Schmitz, H., & Doerr, H. W. (2003) N EngI J Med 348, 1967-1976.
2. Ksiazek, T. G., Erdman, D., Goldsmith, C. S., Zaki, S. R., Peret, T., Emery, S., Tong, S., Urbani, C., Cormer, J. A., Lim, W., Rollin, P. E., Dowell, S. F., Ling, A. E., Humphrey, C. D., Shieh, W. J., Guarner, J., Paddock, C. D., Rota, P., Fields, B., DeRisi, J., Yang, J. Y., Cox, N., Hughes, J. M., LeDuc, J. W., Bellini, W. J., & Anderson, L. J. (2003) N EngI J Med 348, 1953-1966.
3. Peiris, J. S., Lai, S. T., Poon, L. L., Guan, Y., Yam, L. Y., Lim, W., Nicholls, J., Yee, W. K., Yan, W. W., Cheung, M. T., Cheng, V. C., Chan, K. H., Tsang, D. N., Yung, R. W., Ng, T. K., & Yuen, K. Y. (2003) Lancet 361, 1319-1325.
4. Poon, L. L., Wong, O. K., Luk, W., Yuen, K. Y., Peiris, J. S., & Guan, Y. (2003) Clin Chem 49, 953-955.
5. El-Sahly, H. M., Atmar, R. L., Glezen, W. P., & Greenberg, S. B. (2000) Clin Infect Dis 31, 96-100.
6. Folz, R. J. & Elkordy, M. A. (1999) Chest 115, 901-905.
7. Holmes, K. V. (2001) In: D. M. Knipe and P. M Howley, Editors, Fields Virology (4th ed.), Lippincott Williams and Wilkins, Philadelphia 1187-1203.
8. Mcintosh, K. (1974) Current Topics Microbiology Immunology 63, 85-129.
9. Martina, B. E., Haagmans, B. L., Kuiken, T., Fouchier, R. A., Rimmelzwaan, G. F., Van Amerongen, G., Peiris, J. S., Lim, W., & Osterhaus, A. D. (2003) Nature 425, 915.
10. Guan, Y., Zheng, B. J., He, Y. Q., Liu, X. L., Zhuang, Z. X., Cheung, C. L., Luo, S. W., Li, P. H., Zhang, L. J., Guan, Y. J., Butt, K. M., Wong, K. L., Chan, K. W., Lim, W., Shortridge, K. F., Yuen, K. Y., Peiris, J. S., & Poon, L. L. (2003) Science 302, 276-278.
11. Bos, E. C., Heijnen, L., Luytjes, W., & Spaan, W. J. (1995) Virology 214, 453-463.
12. De Groot, R. J., Van Leen, R. W., Dalderup, M. J., Vennema, H., Horzinek, M. C., & Spaan, W. J. (1989) Virology 171, 493-502.
13. Luo, Z. & Weiss, S. R. (1998) Virology 244, 483-494.
14. Spaan, W., Cavanagh, D., & Horzinek, M. C. (1988) J Gen Virol 69 (Pt 12), 2939-2952.
15. Frana, M. F., Behnke, J. N., Sturman, L. S., & Holmes, K. V. (1985) J Virol 56, 912-920.
16. Sturman, L. S., Ricard, C. S., & Holmes, K. V. (1985) J Virol 56, 904-911.
17. Cavanagh, D., Davis, P. J., Darbyshire, J. H., & Peters, R. W. (1986) J Gen Virol 67 (Pt 7), 1435-1442.
18. Taguchi, F. (1995) J Virol 69, 7260-7263.
19. Bosch, B. J., van der Zee, R., de Haan, C. A., & Rottier, P. J. (2003) J Virol 77, 8801-8811.
20. Luo, Z., Matthews, A. M., & Weiss, S. R. (1999) J Virol 73, 8152-8159.
21. Matsuyama, S. & Taguchi, F. (2002) J Virol 76, 11819-11826.
22. Taguchi, F. & Matsuyama, S. (2002) J Virol 76, 950-958.
23. Zelus, B. D., Schickli, J. H., Blau, D. M., Weiss, S. R., & Holmes, K. V. (2003) J Virol 77, 830-840.
24. Skehel, J. J. & Wiley, D. C. (1998) Cell 95, 871-874.
25. Baker, K. A., Dutch, R. E., Lamb, R. A., & Jardetzky, T. S. (1999) Mol Cell 3, 309-319.
26. Chan, D. C., Fass, D., Berger, J. M., & Kim, P. S. (1997) Cell 89, 263-273.
27. Caffrey, M., Cai, M., Kaufman, J., Stahl, S. J., Wingfield, P. T., Covell, D. G., Gronenborn, A. M., & Clore, G. M. (1998) Embo J 17, 4572-4584.
28. Fass, D., Harrison, S. C., & Kim, P. S. (1996) Nat Struct Biol 3, 465-469.
29. Kobe, B., Center, R. J., Kemp, B. E., & Poumbourios, P. (1999) Proc Natl Acad Sci USA 96, 4319-4324.
30. Malashkevich, V. N., Chan, D. C., Chutkowski, C. T., & Kim, P. S. (1998) Proc Natl Acad Sci USA 95, 9134-9139.
31. Malashkevich, V. N., Schneider, B. J., McNally, M. L., Milhollen, M. A., Pang, J. X., & Kim, P. S. (1999) Proc Natl Acad Sci USA 96, 2662-2667.
32. Weissenhorn, W., Dessen, A., Harrison, S. C., Skehel, J. J., & Wiley, D. C. (1997) Nature 387, 426-430.
33. Weissenhorn, W., Carfi, A., Lee, K. H., Skehel, J. J., & Wiley, D.C. (1998) Mol Cell 2, 605-616.
34. Tan, K., Liu, J., Wang, J., Shen, S., & Lu, M. (1997) Proc Natl Acad Sci USA 94, 12303-12308.
35. Yang, Z. N., Mueser, T. C., Kaufman, J., Stahl, S. J., Wingfield, P. T., & Hyde, C. C. (1999) J Struct Biol 126, 131-144.
36. Zhao, X., Singh, M., Malashkevich, V. N., & Kim, P. S. (2000) Proc Natl Acad Sci USA 97, 14172-14177.
37. Bewley, C. A., Louis, J. M., Ghirlando, R., & Clore, G. M. (2002) J Biol Chem 277, 14238-14245.
38. Ghosh, J. K., Peisajovich, S. G., Ovadia, M., & Shai, Y. (1998) J Biol Chem 273, 27182-27190.
39. Medinas, R. J., Lambert, D. M., & Tompkins, W. A. (2002) J Virol 76, 9079-9086.
40. Pinon, J. D., Kelly, S. M., Price, N. C., Flanagan, J. U., & Brighty, D. W. (2003) J Virol 77, 3281-3290.
41. Pritsker, M., Jones, P., Blumenthal, R., & Shai, Y. (1998) Proc Natl Acad Sci USA 95, 7287-7292.
42. Rapaport, D., Ovadia, M., & Shai, Y. (1995) Embo J 14, 5524-5531.
43. Tomasi, M., Pasti, C., Manfrinato, C., Dallocchio, F., & Bellini, T. (2003) FEBS Lett 536, 56-60.
44. Yang, C. & Compans, R. W. (1997) J Virol 71, 8490-8496.
45. Zhou, G., Ferrer, M., Chopra, R., Kapoor, T. M., Strassmaier, T., Weissenhorn, W., Skehel, J. J., Oprian, D., Schreiber, S. L., Harrison, S. C., & Wiley, D. C. (2000) Bioorg Med Chem 8, 2219-2227.
46. Young, J. K., Hicks, R. P., Wright, G. E., & Morrison, T. G. (1997) Virology 238, 291-304.
47. Tripet, B., Wagschal, K., Lavigne, P., Mant, C. T., & Hodges, R. S. (2000) J Mol Biol 300, 377-402.
48. Tabor, S. & Richardson, C. C. (1985) Proc Natl Acad Sci USA 82, 1074-1078.
49. Zelus, B. D., Wessner, D. R., Williams, R. K., Pensiero, M. N., Phibbs, F. T., deSouza, M., Dveksler, G. S., & Holmes, K. V. (1998) J Virol 72, 7237-7244.
50. Hayes, D. B., Laue, T., & Philo, J. (2003) Sedimentation Interpretation program, V 1.08, University of New Hampshire.
51. Johnson, M. L., Correia, J. J., Yphantis, D. A., & Halvorson, H. R. (1981) Biophys J 36, 575-588.
52. Semchuk, P. D., Monera, O. D., Kondejewski, L. H., Gannon, C., Daniels, L., Wilson, I., & Hodges, R. S. (1996) In Peptides: Chemistry, Structure and Biology, P. T. P. Kaumaya and R. S. Hodges (Eds.) Mayflower Scientific Ltd.
53. Tripet, B.& Hodges, R. S. (2001) In Peptides: The Wave of the Future, M. Lebl and R. A. Houghten (Eds.) American Peptide Society.
54. Chen, L., Gorman, J. J., McKimm-Breschkin, J., Lawrence, L. J., Tulloch, P. A., Smith, B. J., Colman, P. M., & Lawrence, M. C. (2001) Structure (Camb) 9, 255-266.
55. Yang, X., Farzan, M., Wyatt, R., & Sodroski, J. (2000) J Virol 74, 5716-5725.
56. Yang, X., Florin, L., Farzan, M., Kolchinsky, P., Kwong, P. D., Sodroski, J., & Wyatt, R. (2000) J Virol 74, 4746-4754.
57. Matthews, J. M., Young, T. F., Tucker, S. P., & Mackay, J. P. (2000) J Virol 74, 5911-5920.
58. Weissenhorn, W., Calder, L. J., Wharton, S. A., Skehel, J. J., & Wiley, D. C. (1998) Proc Natl Acad Sci USA 95, 6032-6036.
59. Gibbons, D. L., Ahn, A., Chatterjee, P. K., & Kielian, M. (2000) J Virol 74, 7772-7780.
60. Bullough, P. A., Hughson, F. M., Skehel, J. J., & Wiley, D. C. (1994) Nature 371, 37-43.
61. Carr, C. M., Chaudhry, C., & Kim, P. S. (1997) Proc Natl Acad Sci USA 94, 14306-14313.
62. Watowich, S. J., Skehel, J. J., & Wiley, D. C. (1994) Structure 2, 719-731.
63. Wilson, I. A., Skehel, J. J., & Wiley, D. C. (1981) Nature 289, 366-373.
64. Harbury, P. B., Zhang, T., Kim, P. S., & Alber, T. (1993) Science 262, 1401-1407.
65. Kohn, W. D., Kay, C. M., & Hodges, R. S. (1998) J Mol Biol 283, 993-1012.
66. Holtzer, M. E., Lovett, E. G., d'Avignon, D. A., & Holtzer, A. (1997) Biophys J 73, 1031-1041.
67. Zhou, N. E., Kay, C. M., & Hodges, R. S. (1992) J Biol Chem 267, 2664-2670.
68. Zhou, N. E., Zhu, B. Y., Kay, C. M., & Hodges, R. S. (1992) Biopolymers 32, 419-426.

### REFERENCES IN CONNECTION WITH EXAMPLE 3

Houston et. al. 1995.
Houston et. al. 1996
Biochem 1996 35 10041-10050
Wagschal et. al. 1999
Tripet et. al. 2000
Kieber-Emmons T., Murali R. and Greene M. I. (1997) Therapeutic peptides and peptidomimetics. Curr. Opin. Biotechnol. 8: 435-441.
Ripka A. S. and Rich D. H. (1998) Peptidomimetic design. Curr. Opin. Chem. Biol. 2: 441-452.
Goodman M. and Ro S. (1995) Peptidomimetics for drug Design. In: Wolff, M. E. (ed.) Burger's Medicinal Chemistry and Drug Discovery, Fifth Edition, Volume 1: Principles and Practice, John Wiley & Sons, Inc., 803-861.

### REFERENCES IN CONNECTION WITH EXAMPLE 5

Lu and Hodges, 2002

### OTHER REFERENCES

Kliger Y and Levanon E Y, Cloaked similarity between HIV-1 and SARS CoV suggests an anti-SARS strategy, BMC Microbiology 2003 Sep 21; 3(1):20. U.S. Pat. No.: 5,464,933 by Bolognesi, et al.; U.S. Pat. Nos. 6,054,2651; 6,541,020; 6,518,013 Tripet B et al., 2004; Structural characterization of the SARS-Coronavirus spike S fusion protein core; J. Biol. Chem. 279 (20): 20836-20849.

### STATEMENTS REGARDING INCORPORATION BY REFERENCE AND VARIATIONS

All references throughout this application, for example publications, patents, and patent documents, are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

Any appendix or appendices hereto are incorporated by reference as part of the specification and/or drawings.

Where the terms “comprise”, “comprises”, “comprised”, or “comprising” are used herein, they are to be interpreted as specifying the presence of the stated features, integers, steps, or components referred to, but not to preclude the presence or addition of one or more other feature, integer, step, component, or group thereof. Separate embodiments of the invention are also intended to be encompassed wherein the terms “comprising” or “comprise(s)” or “comprised” are optionally replaced with the terms, analogous in grammar, e.g.; “consisting/consist(s)” or “consisting essentially of/consist(s) essentially of” to thereby describe further embodiments that are not necessarily coextensive.

The invention has been described with reference to various specific and preferred embodiments and techniques. However, it should be understood that many variations and modifications may be made while remaining within the spirit and scope of the invention. It will be apparent to one of ordinary skill in the art that compositions, methods, devices, device elements, materials, procedures and techniques other than those specifically described herein can be applied to the practice of the invention as broadly disclosed herein without resort to undue experimentation. All art-known functional equivalents of compositions, methods, devices, device elements, materials, procedures and techniques described herein are intended to be encompassed by this invention. Whenever a range is disclosed, all subranges and individual values are intended to be encompassed. This invention is not to be limited by the embodiments disclosed, including any shown in the drawings or exemplified in the specification, which are given by way of example or illustration and not of limitation. The scope of the invention shall be limited only by the claims.

1. A composition comprising a purified peptide of a SARS coronavirus S protein, wherein said peptide is capable of modification of SARS coronavirus infectivity.

2. The composition of claim 1 wherein said modification is an inhibition of infectivity.

3. The composition of claim 1 wherein said peptide has a conformational constraint, wherein said constraint enhances an ability to maintain an alpha-helical conformation.

4. The composition of claim 1 wherein said peptide comprises a lactam bridge.

5. The composition of claim 1 wherein said peptide is selected from the group consisting of: SEQ ID NOS: 4, 6, 8, 10, 12, 14, 16, 18, 20, 22, 24, 26, 28, 30, 32, 34, 36, 38, 40, 42, 44, 46, 47, 48, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102; and a peptide fragment comprising any continuous 14 amino acids thereof of the foregoing peptides.

6. A composition comprising a purified peptide of a SARS coronavirus S protein, wherein said peptide is capable of modifying an ability of said S protein to form or maintain a conformation relating to fusion or entry in a target cell.

7. A composition comprising a purified peptide HR-N10 (SEQ ID NO:24) or HR-N10a (SEQ ID NO:47).

8. A composition comprising a purified peptide HR-C4 (SEQ ID NO:46) or HR-C4a (SEQ ID NO:48).

9. A composition comprising an alpha-helical trimeric conformation of an HR peptide of a coronavirus spike or fusion protein.

10. A composition comprising an alpha-helical trimeric conformation of a purified HR peptide of a SARS coronavirus S protein.

11. The composition of claim 10 wherein said HR peptide is selected from the group consisting of HR-N10 (SEQ ID NO:24), HR-N10a (SEQ ID NO:47), HR-C4 (SEQ ID NO:46) and HR-C4a (SEQ ID NO:48).

12. The composition of claim 10 wherein said HR peptide is HR-N10 (SEQ ID NO:24) or HR-N10a (SEQ ID NO:47).

13. The composition of claim 10 wherein said HR peptide is HR-C4 (SEQ ID NO:46) or HR-C4a (SEQ ID NO:48).

14. A composition comprising an alpha-helical hetero-trimeric (6-helix) conformation of a purified HR peptide of a coronavirus spike or fusion protein.

15. A composition comprising an alpha-helical hetero-trimeric (6-helix) conformation of a complex comprising a purified HR-N peptide and a purified HR-C peptide of a SARS coronavirus S protein.

16. The composition of claim 15 wherein said HR-N peptide is selected from the group consisting of HR-N1 (SEQ ID NO:6), HR-N2 (SEQ ID NO:8), HR-N10 (SEQ ID NO:24), and HR-N10a (SEQ ID NO:47); and said HR-C peptide is selected from the group consisting of HR-C1 (SEQ ID NO:40), HR-C4 (SEQ ID NO:46), and HR-C4a (SEQ ID NO:48).

17. The composition of claim 15 wherein said HR-N peptide is HR-N10 (SEQ ID NO:24) or HR-N10a (SEQ ID NO:47) and said HR-C peptide is HR-C4 (SEQ ID NO:46) or HR-C4a (SEQ ID NO:48).

18. The composition of claim 16 wherein said HR-N peptide is HR-N10 (SEQ ID NO:24) or HR-N10a (SEQ ID NO:47) and said HR-C peptide is HR-C1 (SEQ ID NO:40).

19. A composition comprising a purified HR-N or HR-C peptide of SARS coronavirus S protein, wherein said peptide is capable of modification of SARS coronavirus infectivity and wherein said peptide has a transition midpoint temperature of from about 35 to about 90 degrees Celsius.

20. The composition of claim 19 wherein said temperature is from about 36 to about 74 degrees Celsius.

21. The composition of claim 19 wherein said temperature is from about 37 to about 60 degrees Celsius.

22. The composition of claim 19 wherein said temperature is from about 56 to about 57 degrees Celsius.

23. A purified peptide comprising at least about 14 contiguous amino acids derived from a peptide selected from the group consisting of: HR-N10 (SEQ ID NO:24), HR-N10a (SEQ ID NO:47), HR-C4 (SEQ ID NO:46), and HR-C4a (SEQ ID NO:48), wherein said peptide exhibits a stable helix conformation at a physiological temperature of a human or other mammal.

24. A composition comprising a purified nucleic acid molecule encoding a peptide of SARS coronavirus S protein, wherein said peptide is capable of modification of SARS coronavirus infectivity.

25. A composition comprising a purified nucleic acid molecule encoding an HR peptide of a SARS coronavirus S protein.

26. A composition comprising a purified nucleic acid molecule capable of encoding a peptide selected from the group consisting of: HR-N10 (SEQ ID NO:24), HR-N10a (SEQ ID NO:47), HR-C4 (SEQ ID NO:46), and HR-C4a (SEQ ID NO:48).

27. A composition comprising a purified nucleic acid molecule capable of encoding peptide HR-N10 (SEQ ID NO:24) or HR-N10a (SEQ ID NO:47).

28. A composition comprising a purified nucleic acid molecule capable of encoding peptide HR-C4 (SEQ ID NO:46) or HR-C4a (SEQ ID NO:48).

29. A method of identifying a peptide composition capable of inhibiting a SARS coronavirus, comprising: providing a peptide of an HR-N or HR-C domain of a SARS coronavirus S protein; and measuring an ability of said peptide to inhibit infectivity of a SARS coronavirus or cell fusion in connection with a SARS coronavirus protein; thereby identifying a peptide composition capable of inhibiting a SARS coronavirus.

30. A method of treating a SARS coronavirus infection in a human subject, comprising administering an effective amount of a peptide of a SARS coronavirus S protein.

31. A method of treating a SARS coronavirus infection in a human subject, comprising administering an effective amount of an antibody reactive towards a material selected from the group consisting of a SARS coronavirus S protein, a peptide of a SARS coronavirus S protein, an HR-N peptide of a SARS coronavirus S protein, an HR-C peptide of a SARS coronavirus S protein, HR-N10, HR-N10a, HR-C4, HR-C4a, HR-C1, a trimeric conformation of a SARS coronavirus peptide, a six helix bundle conformation of a complex of a SARS coronavirus HR-N peptide and an HR-C peptide.

32. The method of claim 31 wherein said material is HR-N10 or HR-N10a.

33. The method of claim 31 wherein said material is HR-C4 or HR-C4a.

34. The method of claim 31 wherein said material comprises an HR-N peptide and an HR-C peptide.

35. The method of claim 31 wherein said material comprises an HR-N peptide of HR-N10 or HR-N10a and an HR-C peptide of HR-C4 or HR-C4a.

36. The method of claim 31 wherein said material comprises an HR-N peptide of HR-N10 or HR-N10a and an HR-C peptide of HR-C1.

37. A SARS coronavirus purified peptide composition capable of stimulating an immune response, wherein said composition is selected from the group consisting of: a SARS coronavirus S protein; a peptide of a SARS coronavirus S protein; an HR-N peptide of a SARS coronavirus S protein; an HR-C peptide of a SARS coronavirus S protein; HR-N10; HR-N10a; HR-C4; HR-C4a; HR-C1; a trimeric conformation of a SARS coronavirus peptide; a six helix bundle conformation of a complex of a SARS coronavirus HR-N peptide and an HR-C peptide; and a peptide fragment comprising any continuous 14 amino acids thereof of the foregoing peptides.

38. A method of identifying or screening for an inhibitory peptide of a SARS coronavirus, comprising: providing a synthetic or recombinant peptide of HR-N or HR-C; evaluating said peptide for an ability to make a structure selected from the group consisting of an alpha-helical coil, an alpha-helical trimer, and an alpha-helical hetero-trimeric (6-helix) bundle; testing said peptide in an infectivity or cell-cell fusion bioassay, wherein a first result where said peptide is present and demonstrates a reduced infectivity or fusion in comparison with a second result where said peptide is absent; thereby identifying or screening for an inhibitory peptide.

39. The composition of claim 1 wherein said peptide is an HR-N peptide selected from the group consisting of HR-N10 (SEQ ID NO:24) and HR-N10a (SEQ ID NO:47).

40. The composition of claim 1 wherein said peptide is an HR-C peptide selected from the group consisting of HR-C4 (SEQ ID NO:46) and HR-C4a (SEQ ID NO:48).


SEQUENCE LISTING (106 sequences)

SEQ ID NO: 1 - DNA, 3768 bp, SARS coronavirus Urbani
  CDS (1)..(3768)
atgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagctcgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataa

SEQ ID NO: 2 - protein, 1255 aa, SARS coronavirus Urbani
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 3 - DNA, 390 bp, SARS coronavirus Urbani
  CDS (1)..(390)
atgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatg

SEQ ID NO: 4 - protein, 130 aa, SARS coronavirus Urbani
MQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKM

SEQ ID NO: 5 - DNA, 276 bp, SARS coronavirus Urbani
  CDS (1)..(276)
atgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggta

SEQ ID NO: 6 - protein, 92 aa, SARS coronavirus Urbani
MQMAYRFNGIGVTQNVLYENQKQIANQFNKAISQIQESLTTTSTALGKLQDVVNQNAQALNTLVKQLSSNFGAISSVLNDILSRLDKVEAEV

SEQ ID NO: 7 - DNA, 174 bp, SARS coronavirus Urbani
  CDS (1)..(174)
attcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggta

SEQ ID NO: 8 - protein, 58 aa, SARS coronavirus Urbani
IQESLTTTSTALGKLQDVVNQNAQALNTLVKQLSSNFGAISSVLNDILSRLDKVEAEV

SEQ ID NO: 9 - DNA, 141 bp, SARS coronavirus Urbani
  CDS (1)..(141)
ttgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggta

SEQ ID NO: 10 - protein, 47 aa, SARS coronavirus Urbani
LGKLQDVVNQNAQALNTLVKQLSSNFGAISSVLNDILSRLDKVEAEV

SEQ ID NO: 11 - DNA, 114 bp, SARS coronavirus Urbani
  CDS (1)..(114)
caaattgacaggttaattacaggcagacttcaaagccttcaaacctatgtaacacaacaactaatcagggctgctgaaatcagggcttctgctaatcttgctgctactaaaatg

SEQ ID NO: 12 - protein, 38 aa, SARS coronavirus Urbani
QIDRLITGRLQSLQTYVTQQLIRAAEIRASANLAATKM

SEQ ID NO: 13 - DNA, 105 bp, SARS coronavirus Urbani
  CDS (1)..(105)
atgcaaatggcatataggttcaatggcattggagttacccaaaatgttctctatgagaaccaaaaacaaatcgccaaccaatttaacaaggcgattagtcaaatt

SEQ ID NO: 14 - protein, 35 aa, SARS coronavirus Urbani
MQMAYRFNGIGVTQNVLYENQKQIANQFNKAISQI

SEQ ID NO: 15 - DNA, 105 bp, SARS coronavirus Urbani
  CDS (1)..(105)
ttcaatggcattggagttacccaaaatgttctctatgagaaccaaaaacaaatcgccaaccaatttaacaaggcgattagtcaaattcaagaatcacttacaaca

SEQ ID NO: 16 - protein, 35 aa, SARS coronavirus Urbani
FNGIGVTQNVLYENQKQIANQFNKAISQIQESLTT

SEQ ID NO: 17 - DNA, 105 bp, SARS coronavirus Urbani
  CDS (1)..(105)
caaaatgttctctatgagaaccaaaaacaaatcgccaaccaatttaacaaggcgattagtcaaattcaagaatcacttacaacaacatcaactgcattgggcaag

SEQ ID NO: 18 - protein, 35 aa, SARS coronavirus Urbani
QNVLYENQKQIANQFNKAISQIQESLTTTSTALGK

SEQ ID NO: 19 - DNA, 105 bp, SARS coronavirus Urbani
  CDS (1)..(105)
caaaaacaaatcgccaaccaatttaacaaggcgattagtcaaattcaagaatcacttacaacaacatcaactgcattgggcaagctgcaagacgttgttaaccag

SEQ ID NO: 20 - protein, 35 aa, SARS coronavirus Urbani
QKQIANQFNKAISQIQESLTTTSTALGKLQDVVNQ

SEQ ID NO: 21 - DNA, 105 bp, SARS coronavirus Urbani
  CDS (1)..(105)
tttaacaaggcgattagtcaaattcaagaatcacttacaacaacatcaactgcattgggcaagctgcaagacgttgttaaccagaatgctcaagcattaaacaca

SEQ ID NO: 22 - protein, 35 aa, SARS coronavirus Urbani
FNKAISQIQESLTTTSTALGKLQDVVNQNAQALNT

SEQ ID NO: 23 - DNA, 105 bp, SARS coronavirus Urbani
  CDS (1)..(105)
attcaagaatcacttacaacaacatcaactgcattgggcaagctgcaagacgttgttaaccagaatgctcaagcattaaacacacttgttaaacaacttagctct

SEQ ID NO: 24 - protein, 35 aa, SARS coronavirus Urbani
IQESLTTTSTALGKLQDVVNQNAQALNTLVKQLSS

SEQ ID NO: 25 - DNA, 105 bp, SARS coronavirus Urbani
  CDS (1)..(105)
acatcaactgcattgggcaagctgcaagacgttgttaaccagaatgctcaagcattaaacacacttgttaaacaacttagctctaattttggtgcaatttcaagt

SEQ ID NO: 26 - protein, 35 aa, SARS coronavirus Urbani
TSTALGKLQDVVNQNAQALNTLVKQLSSNFGAISS

SEQ ID NO: 27 - DNA, 105 bp, SARS coronavirus Urbani
  CDS (1)..(105)
caagacgttgttaaccagaatgctcaagcattaaacacacttgttaaacaacttagctctaattttggtgcaatttcaagtgtgctaaatgatatcctttcgcga

SEQ ID NO: 28 - protein, 35 aa, SARS coronavirus Urbani
QDVVNQNAQALNTLVKQLSSNFGAISSVLNDILSR

SEQ ID NO: 29 - DNA, 105 bp, SARS coronavirus Urbani
  CDS (1)..(105)
gctcaagcattaaacacacttgttaaacaacttagctctaattttggtgcaatttcaagtgtgctaaatgatatcctttcgcgacttgataaagtcgaggcggag

SEQ ID NO: 30 - protein, 35 aa, SARS coronavirus Urbani
AQALNTLVKQLSSNFGAISSVLNDILSRLDKVEAE

SEQ ID NO: 31 - DNA, 105 bp, SARS coronavirus Urbani
  CDS (1)..(105)
gttaaacaacttagctctaattttggtgcaatttcaagtgtgctaaatgatatcctttcgcgacttgataaagtcgaggcggaggtacaaattgacaggttaatt

SEQ ID NO: 32 - protein, 35 aa, SARS coronavirus Urbani
VKQLSSNFGAISSVLNDILSRLDKVEAEVQIDRLI

SEQ ID NO: 33 - DNA, 105 bp, SARS coronavirus Urbani
  CDS (1)..(105)
tttggtgcaatttcaagtgtgctaaatgatatcctttcgcgacttgataaagtcgaggcggaggtacaaattgacaggttaattacaggcagacttcaaagcctt

SEQ ID NO: 34 - protein, 35 aa, SARS coronavirus Urbani
FGAISSVLNDILSRLDKVEAEVQIDRLITGRLQSL

SEQ ID NO: 35 - DNA, 105 bp, SARS coronavirus Urbani
  CDS (1)..(105)
ctaaatgatatcctttcgcgacttgataaagtcgaggcggaggtacaaattgacaggttaattacaggcagacttcaaagccttcaaacctatgtaacacaacaa

SEQ ID NO: 36 - protein, 35 aa, SARS coronavirus Urbani
LNDILSRLDKVEAEVQIDRLITGRLQSLQTYVTQQ

SEQ ID NO: 37 - DNA, 105 bp, SARS coronavirus Urbani
  CDS (1)..(105)
cttgataaagtcgaggcggaggtacaaattgacaggttaattacaggcagacttcaaagccttcaaacctatgtaacacaacaactaatcagggctgctgaaatc

SEQ ID NO: 38 - protein, 35 aa, SARS coronavirus Urbani
LDKVEAEVQIDRLITGRLQSLQTYVTQQLIRAAEI

SEQ ID NO: 39 - DNA, 123 bp, SARS coronavirus Urbani
  CDS (1)..(123)
gatgttgatcttggcgacatttcaggcattaacgcttctgtcgtcaacattcaaaaagaaattgaccgcctcaatgaggtcgctaaaaatttaaatgaatcactcattgaccttcaagaattg

SEQ ID NO: 40 - protein, 41 aa, SARS coronavirus Urbani
DVDLGDISGINASVVNIQKEIDRLNEVAKNLNESLIDLQEL

SEQ ID NO: 41 - DNA, 63 bp, SARS coronavirus Urbani
  CDS (1)..(63)
attgaccgcctcaatgaggtcgctaaaaatttaaatgaatcactcattgaccttcaagaattg

SEQ ID NO: 42 - protein, 21 aa, SARS coronavirus Urbani
IDRLNEVAKNLNESLIDLQEL

SEQ ID NO: 43 - DNA, 84 bp, SARS coronavirus Urbani
  CDS (1)..(84)
gtcgtcaacattcaaaaagaaattgaccgcctcaatgaggtcgctaaaaatttaaatgaatcactcattgaccttcaagaattg

SEQ ID NO: 44 - protein, 28 aa, SARS coronavirus Urbani
VVNIQKEIDRLNEVAKNLNESLIDLQEL

SEQ ID NO: 45 - DNA, 105 bp, SARS coronavirus Urbani
  CDS (1)..(105)
atttcaggcattaacgcttctgtcgtcaacattcaaaaagaaattgaccgcctcaatgaggtcgctaaaaatttaaatgaatcactcattgaccttcaagaattg

SEQ ID NO: 46 - protein, 35 aa, SARS coronavirus Urbani
ISGINASVVNIQKEIDRLNEVAKNLNESLIDLQEL

SEQ ID NO: 47 - protein, 49 aa, SARS coronavirus Urbani
QKQIANQFNKAISQIQESLTTTSTALGKLQDVVNQNAQALNTLVKQLSS

SEQ ID NO: 48 - protein, 36 aa, SARS coronavirus Urbani
DISGINASVVNIQKEIDRLNEVAKNLNESLIDLQEL

SEQ ID NO: 49 - protein, 1255 aa, SARS coronavirus Urbani; identical to SEQ ID NO: 2

SEQ ID NO: 50 - protein, 35 aa, SARS coronavirus Urbani; identical to SEQ ID NO: 24

SEQ ID NO: 51 - protein, 35 aa, Artificial
  Synthetic peptide
IQAALTKTSAALGKLQAAVNRNAAALNKLVKALSS

SEQ ID NO: 52 - protein, 35 aa, Artificial
  Synthetic peptide
IQESLTXTSTALGKLQDVVNXNAQALNXLVKQLSS

SEQ ID NO: 53 - protein, 35 aa, Artificial; identical to SEQ ID NO: 52
  Synthetic peptide

SEQ ID NO: 54 - protein, 35 aa, Artificial
  Synthetic peptide
IQESLTTTSTALGKLQEVVNKNAQALNTLVKQLSS

SEQ ID NO: 55 - protein, 35 aa, Artificial
  Synthetic peptide
IQESLTETSTKLGKLQDVVNQNAQALNELVKKLSS

SEQ ID NO: 56 - protein, 35 aa, Artificial
  Synthetic peptide
IQESLTTTSTALGELQDVVNENAQALNTLVKQLSS

SEQ ID NO: 57 - protein, 35 aa, Artificial
  Synthetic peptide
IIESLTTTITALGKLIDVLNQNIQALNTLIKQLSS

SEQ ID NO: 58 - protein, 35 aa, SARS coronavirus Urbani; identical to SEQ ID NO: 46

SEQ ID NO: 59 - protein, 35 aa, Artificial
  Synthetic peptide
IAAINKSVAAIQKEIARLNEVAKALNASLIRLQAL

SEQ ID NO: 60 - protein, 35 aa, Artificial
  Synthetic peptide
ISGINXSVVNIQKEIDRLNXVAKNLNXSLIDLQEL

SEQ ID NO: 61 - protein, 35 aa, Artificial; identical to SEQ ID NO: 60
  Synthetic peptide

SEQ ID NO: 62 - protein, 35 aa, Artificial
  Synthetic peptide
ISGINASVVNIQKEIERLNKVAKNLNESLIDLQEL

SEQ ID NO: 63 - protein, 35 aa, Artificial
  Synthetic peptide
ISGINESVVKIQKEIDRLNEVAKNLNESLIKLQEL

SEQ ID NO: 64 - protein, 35 aa, Artificial
  Synthetic peptide
ISGINASVVNIQEEIDRLNEVAKNLNESLIDLQEL

SEQ ID NO: 65 - protein, 35 aa, Artificial
  Synthetic peptide
ISGINASIVNIQKEIDRLNEVIKNLNESLIDLQEL

SEQ ID NO: 66 - protein, 39 aa, SARS coronavirus Urbani
DLGDISGINASVVNIQKEIDRLNEVAKNLNESLIDLQEL

SEQ ID NO: 67 - protein, 36 aa, Artificial
  Synthetic peptide
DISGINASVVNIQKEIDRLNEVIKNLNESLIDLQEL

SEQ ID NO: 68 - protein, 36 aa, Artificial
  Synthetic peptide
DISGINASVVNIQKEIARLNEVAKALNESLIDLQEL

SEQ ID NO: 69 - protein, 36 aa, Artificial
  Synthetic peptide
DISGINASVVNIQKEIARLNEVIKALNESLIDLQEL

SEQ ID NO: 70 - protein, 36 aa, Artificial
  Synthetic peptide
DIAAINASVANIQKEIARLNEVAKALNESLAALQAL

SEQ ID NO: 71 - protein, 36 aa, Artificial
  Synthetic peptide
DISGINASVVNIQKEIERLNKVAKNLNESLIDLQEL

SEQ ID NO: 72 - protein, 36 aa, Artificial; identical to SEQ ID NO: 71
  Synthetic peptide

SEQ ID NO: 73 - protein, 36 aa, Artificial
  Synthetic peptide
DIEEINKKVEEIQKKIEELNKKAEELNKKLEELQKK

SEQ ID NO: 74 - protein, 36 aa, Artificial
  Synthetic peptide
DISGINASVVEIQKKIEELNKKAEELNKKLIDLQEL

SEQ ID NO: 75 - protein, 7 aa, Artificial
  Synthetic peptide
IQESLTT

SEQ ID NO: 76 - protein, 7 aa, Artificial
  Synthetic peptide
TSTALGK

SEQ ID NO: 77 - protein, 7 aa, Artificial
  Synthetic peptide
LQDVVNQ

SEQ ID NO: 78 - protein, 7 aa, Artificial
  Synthetic peptide
NAQALNT

SEQ ID NO: 79 - protein, 7 aa, Artificial
  Synthetic peptide
LVKQLSS

SEQ ID NO: 80 - protein, 14 aa, Artificial
  Synthetic peptide
IQESLTTTSTALGK

SEQ ID NO: 81 - protein, 14 aa, Artificial
  Synthetic peptide
TSTALGKLQDVVNQ

SEQ ID NO: 82 - protein, 14 aa, Artificial
  Synthetic peptide
LQDVVNQNAQALNT

SEQ ID NO: 83 - protein, 14 aa, Artificial
  Synthetic peptide
NAQALNTLVKQLSS

SEQ ID NO: 84 - protein, 21 aa, Artificial
  Synthetic peptide
IQESLTTTSTALGKLQDVVNQ

SEQ ID NO: 85 - protein, 21 aa, Artificial
  Synthetic peptide
TSTALGKLQDVVNQNAQALNT

SEQ ID NO: 86 - protein, 21 aa, Artificial
  Synthetic peptide
LQDVVNQNAQALNTLVKQLSS

SEQ ID NO: 87 - protein, 28 aa, Artificial
  Synthetic peptide
IQESLTTTSTALGKLQDVVNQNAQALNT

SEQ ID NO: 88 - protein, 28 aa, Artificial
  Synthetic peptide
TSTALGKLQDVVNQNAQALNTLVKQLSS

SEQ ID NO: 89 - protein, 7 aa, Artificial
  Synthetic peptide
ISGINAS

SEQ ID NO: 90 - protein, 7 aa, Artificial
  Synthetic peptide
VVNIQKE

SEQ ID NO: 91 - protein, 7 aa, Artificial
  Synthetic peptide
IDRLNEV

SEQ ID NO: 92 - protein, 7 aa, Artificial
  Synthetic peptide
AKNLNES

SEQ ID NO: 93 - protein, 7 aa, Artificial
  Synthetic peptide
LIDLQEL

SEQ ID NO: 94 - protein, 14 aa, Artificial
  Synthetic peptide
ISGINASVVNIQKE

SEQ ID NO: 95 - protein, 14 aa, Artificial
  Synthetic peptide
VVNIQKEIDRLNEV

SEQ ID NO: 96 - protein, 14 aa, Artificial
  Synthetic peptide
IDRLNEVAKNLNES

SEQ ID NO: 97 - protein, 14 aa, Artificial
  Synthetic peptide
AKNLNESLIDLQEL

SEQ ID NO: 98 - protein, 21 aa, Artificial
  Synthetic peptide
ISGINASVVNIQKEIDRLNEV

SEQ ID NO: 99 - protein, 21 aa, Artificial
  Synthetic peptide
VVNIQKEIDRLNEVAKNLNES

SEQ ID NO: 100 - protein, 21 aa, Artificial
  Synthetic peptide
IDRLNEVAKNLNESLIDLQEL

SEQ ID NO: 101 - protein, 28 aa, Artificial
  Synthetic peptide
ISGINASVVNIQKEIDRLNEVAKNLNES

SEQ ID NO: 102 - protein, 28 aa, Artificial
  Synthetic peptide
VVNIQKEIDRLNEVAKNLNESLIDLQEL

SEQ ID NO: 103 - DNA, 36 bp, Artificial
  Synthetic construct
gtacgtacgcatatgatgcaaatggcatataggttc

SEQ ID NO: 104 - DNA, 58 bp, Artificial
  Synthetic construct
gcgaattccctttgtcgtcgtcgtcgcagccgcctacctccgcctcgactttatcaag

SEQ ID NO: 105 - DNA, 25 bp, Artificial
  Synthetic construct
caccatgtttattttcttattattt

SEQ ID NO: 106 - DNA, 24 bp, Artificial
  Synthetic construct
ttatgtgtaatgtaatttgacacc